(19) United States
(12) Patent Application Publication
(10) Pub. No.: US 2024/0237973 A1
(43) Pub. Date: Jul. 18, 2024

(54) INTESTINAL CAPSULE ASSEMBLY

(71) Applicant: VESTLANDETS INNOVASJONSSELSKAP AS, Bergen (NO)

(72) Inventors: Hans Steinsland, Bergen (NO); Halvor Sommerfelt, Bergen (NO)

(21) Appl. No.: 18/560,251

(22) PCT Filed: May 6, 2022
(86) PCT No.: PCT/EP2022/062377

§ 371 Date: Nov. 10, 2023

(30) Foreign Application Priority Data
May 10, 2021 (GB) 2106647.7

(51) Int. Cl. A61B 10/00

(52) U.S. Cl. CPC A61B 10/0045; A61B 2010/0061

(57) ABSTRACT

An intestinal capsule assembly includes a housing. The housing has a first opening, and a sample chamber 2 defining a volume for collecting a sample of intestinal fluid. The assembly also includes a releasable module, comprising a battery and a fluid container, retained by a retaining mechanism. In a pre-activation configuration, the releasable module is located within the housing. In a post-activation configuration, the releasable module is located outside of the housing. The releasable module is arranged to be activated, to cause the battery to supply power to the fluid container so as to cause, in use, electrolysis of a fluid within the fluid container to create a gas pressure, wherein the gas pressure causes release of the retaining mechanism, so as to allow movement of the releasable module out of the housing through the first opening, resulting in expansion of the volume of the sample chamber, thereby filling it with the fluid sample.

### BACKGROUND OF THE INVENTION

This invention relates to a capsule assembly for retrieving viscous fluid specimens from humans and animals e.g. from the gut lumen including viscous fluids from deep within the small intestine.

The gut is responsible for many of the body's most important functions including nutrient and mineral uptake, adjusting body fluid balance, and defending against potentially harmful microorganisms. Fluids collected from the gut can, for example, be used for research in gut function and for diagnosing and monitoring gastrointestinal disorders and illnesses.

It is a known possibility to collect specimens from deep within the small intestine by using hollow tubes that are threaded through the small intestine to the region of interest. However, such procedures are highly invasive and usually require general anaesthesia. To safely and non-invasively collect fluids from deep within the small intestine there is therefore a need for untethered, ingestible devices that move naturally through the gut to collect specimens.

Since gut disorders and illnesses are increasingly being seen to be important contributors to human and animal ill health, making these devices available to gastroenterologists and other researchers becomes ever more important.

Despite efforts over the past several decades, no known designs for untethered ingestible sampling devices have been made commercially available, and few appear to ever have been produced or tested. Such known designs suffer from a wide range of drawbacks, such as being mechanically infeasible, inherently leaky, too complex to be efficiently produced, or that their functions demand more energy than is currently possible to incorporate in these small devices. Another challenge often not adequately addressed in known designs is that gut fluids are often highly viscous and, therefore, not easily sampled.

The present invention seeks to address at least some of these and other shortcomings of known designs.

### SUMMARY OF THE INVENTION

From a first aspect, the invention provides an intestinal capsule assembly, comprising:
a housing, the housing comprising a first opening, and a sample chamber defining a volume for collecting a sample of intestinal fluid;
a releasable module, comprising a battery and a fluid container; and
a retaining mechanism, for retaining the releasable module within the housing such that the releasable module closes the first opening;
wherein the intestinal capsule assembly has a first, pre-activation configuration in which the releasable module is located within the housing and a second, post-activation configuration, in which the releasable module is located outside of the housing; and
wherein the releasable module is arranged to be activated, to cause the battery to supply power to the fluid container so as to cause, in use, electrolysis of a fluid within the fluid container to create a gas pressure, wherein the gas pressure causes release of the retaining mechanism, so as to allow movement of the releasable module out of the housing through the first opening, resulting in expansion of the volume of the sample chamber.

Thus it will be seen that, in accordance with the invention, by using electrolysis of a fluid to create a gas pressure, where this gas pressure causes release of the retaining mechanism, a simple mechanism to release the releasable module is achieved. Creating the gas pressure needed to release the retaining mechanism requires only low voltages and little electrical energy, which is advantageous since this allows for the use of smaller batteries to trigger sample collection, meaning that the size of the releasable module (and therefore the whole capsule assembly) can be made small enough to be easily ingested, even for children and smaller animals. The larger the intestinal capsule assembly, the more likely it is to cause discomfort to a subject as it is swallowed.

It will be understood that supply of power by the battery, which causes electrolysis, is actively triggered and can therefore be controlled. Thus, in other words, the releasable module is arranged to be activated, to cause the battery to controllably supply power to the fluid container so as to cause, in use, electrolysis of a fluid within the fluid container to create a gas pressure. This differs from, for example, battery gassing, which causes a battery itself to produce gas, but which occurs due to chemical decomposition of battery electrolytes and is therefore not easily actively controllable.

The release of the releasable module allows it to move out of the housing, through the first opening, which allows expansion of the sample chamber so that a sample can be drawn in, and so that the volume of the sample chamber is able to expand into the space which was previously occupied by the releasable module.

Simply releasing the retaining mechanism, which retains the releasable module within the housing, may be sufficient to result in the releasable module moving out of the housing, and as a result fluid being drawn into the sample chamber, e.g. due to lower pressure inside the sample chamber. However, in some embodiments, at least one ejection mechanism is provided which applies a force to the releasable module which results in the releasable module being ejected from the housing. This helps to ensure that the releasable module moves completely out of the housing and thereby triggers the sample chamber to start collecting a sample of intestinal fluid. In some embodiments, the ejection mechanism comprises a resilient biasing member such as a spring.

In some embodiments, the intestinal capsule assembly further comprises a piston, wherein the piston defines a wall of the sample chamber and the expansion of the volume of the sample chamber causes movement of the piston towards the first opening. This provides a simple mechanism which allows the size of the sample chamber to be changed, e.g. to expand into the space previously occupied by the releasable module. In some embodiments, the piston is arranged to seal against a wall of the housing. In some embodiments, the housing may be substantially cylindrical. The piston may have a circular face and may be arranged to seal against an inner wall of the cylindrical housing. In some embodiments, the piston is formed integrally with the releasable module. This reduces the number of separate components within the capsule assembly. In such embodiments, the releasable module would protrude through the first opening when the sample collection has completed.

In some embodiments, the housing is arranged to stop the piston from moving out of the housing through the first opening; thus in some embodiments the housing further comprises a stop. The housing may comprise a lip around the first opening, said lip may provide a stop, or may be additional to a stop. This helps to ensure that the sample chamber, defined in part by the piston, remains sealed and conveniently arranged within the housing, which protects the sample chamber. An inner wall of the housing may define a wall of the sample chamber.

The piston may be moved simply due to pressure differentials which arise as the releasable module moves out of the first opening, but gut fluids are often viscous and will therefore resist being drawn into the sample chamber. It is thus preferable that the piston is biased towards the first opening, such that once the releasable module begins moving out of the first opening, the biasing of the piston will result in expansion of the sample chamber, moving the piston towards the first opening. For example, the piston may be resiliently biased or spring-loaded, i.e. biased by a spring, e.g. a coil spring.

The biasing of the piston, and the ejection mechanism, may be provided by means of two separate mechanisms or biasing members. However it has been appreciated that a single spring may be advantageously utilised to fulfil both of these functions. Thus, in some embodiments, the piston is resiliently biased and the piston provides an ejection mechanism for the releasable module which applies a force to the releasable module which results in the releasable module being ejected from the housing through the first opening, moving the intestinal capsule assembly to the post-activation configuration.

As described above, in some embodiments the housing is arranged to stop the piston from moving out of the housing through the first opening. This thereby defines the position of maximum movement of the piston in a direction towards the first opening, which may be referred to as the stop position. When the piston is in the stop position, this may define the maximum volume of the sample chamber. The movement of the releasable module out of the first opening (and the optional ejection mechanism and/or biasing of the piston) may be sufficient to cause expansion of the sample chamber so as to move the piston all the way to the stop position. However, these mechanisms may be sufficient only to move the piston part of the way to the stop position e.g. if the force applied by a biasing spring is reduced the further it expands. Thus further means may advantageously be provided which assist in moving the piston to the stop position, so that maximum expansion of the sample chamber is achieved. This may, for example, help to provide an effective seal between the piston and the housing e.g. where the seal of the piston is most effective at the stop position. In some embodiments, the piston may seal against a lip of the housing, when in the stop position.

In some embodiments, the intestinal capsule module further comprises a flexible attachment, connecting the piston and the releasable module. The connection of the flexible attachment to the piston may be direct or indirect e.g. there may be an intermediate component attached to both the piston and the flexible attachment. Once the releasable module has been ejected from the housing, each will move independently. In use, when the intestinal module assembly is moving through the intestine, the releasable module and the housing, once separated, will therefore be acted on independently by the peristalsis action of the intestine. At certain points peristalsis will happen to act in opposite directions on the releasable module and the housing, pulling them away from each other. The flexible attachment in this case will pull on the piston, drawing it towards the stop position. As the attachment is flexible, when the housing and the releasable module subsequently happen to move closer together, the attachment will simply flex, or go slack, and the piston will stay in its new position, further towards the stop position. This drawing of the piston towards the stop position helps to harvest the action of peristalsis to help expand the sample chamber as much as possible, thereby maximising the size of the sample collected and helping to ensure consistent sample collection. The flexible attachment may be a cord or thread.

Another means, which may be used additionally or alternatively, to assist in moving the piston to the stop position, so that maximum expansion of the sample chamber is achieved, is a gas generation means within the sample chamber. Thus, in some embodiments, the sample chamber comprises gas generation means, arranged to be triggered, in use, by contact with a sample of intestinal fluid within the sample chamber, to generate gas. This generated gas increases the pressure within the sample chamber, helping to push the piston further towards the stop position. This may help to ensure the piston is in a fully sealed position over the first opening. Optionally the gas generation means may comprise a gas generating medium, such as beads, e.g. gas-generating chemicals compressed into beads.

The beads, or other gas generation means, may be provided with a coating which delays gas generation, e.g. a coating which is dissolved by intestinal fluid over a relatively long period, before gas generation occurs. This means that the gas generation means are only activated if a certain time has already elapsed, which will likely be after other forces acting on the sample chamber have become stabilised or balanced e.g. the gas generation means are activated if or when other means have failed to successfully move the piston all the way to the stop position so as to fill and/or seal the sample chamber.

Electrolysis of a fluid within the (sealed) fluid container creates a gas pressure. In some embodiments, the fluid container comprises an electrolysis solution. The electrolysis solution may contain (buffered) formic acid. In some embodiments, the fluid container comprises electrodes e.g. platinum-based catalyst-coated electrodes, arranged to contact the battery and, in use, to contact an electrolysis solution within the fluid container.

The gas pressure may cause release of the retaining mechanism in any suitable manner, so as to allow movement of the releasable module out of the housing. In some embodiments, the gas pressure in the fluid container causes movement of at least a part of the retaining mechanism.

In some embodiments, the retaining mechanism comprises one or more locking members arranged between the releasable module and the housing, wherein the one or more locking members is moveable from an engaging position in which it is engaged with the housing to retain the releasable module within the housing, to a non-engaging position in which the releasable module is movable relative to the housing. The locking members may be engaged, in the engaging position, with a lip around the first opening. The one or more locking members could be at least one locking pin or ball, optionally multiple locking balls e.g. four, five, or six.

In some embodiments, the retaining mechanism comprises one or more locking members arranged on a surface of the releasable module, wherein the one or more locking members is moveable from an engaging position in which it is engaged with the housing to retain the releasable module within the housing, to a non-engaging position in which the releasable module is movable relative to the housing. For example, the one or more locking members may comprise a protrusion or ridge extending from an external (or outer) surface of the releasable module.

The one or more locking members may form part of the releasable module e.g. multiple locking members may be arranged, optionally at regular intervals, around an outer surface of the releasable module. Thus, in some embodiments, the retaining mechanism comprises one or more locking members formed as part of the releasable module, wherein the one or more locking members is moveable from an engaging position in which it is engaged with the housing to retain the releasable module within the housing, to a non-engaging position in which the releasable module is movable relative to the housing; and wherein the retaining mechanism comprises a release member arranged to be moved by the gas pressure to allow the one or more locking members to move to the non-engaging position. As above, the one or more locking members may be arranged between the releasable module and the housing. It is advantageous that the one or more locking members is part of the releasable module since this may simplify the design of the housing, and also minimizes the number, or volume, of components, which remain within the housing after the releasable module is ejected, thus maximising the volume available for the sample chamber to expand in to. The housing may comprise at least one indent or groove, arranged to accommodate the one or more locking members. There may be multiple such indents or grooves, each arranged to accommodate a respective locking member.

The one or more locking members could be movable directly by the gas pressure created in the fluid container, or they could be movable indirectly as a result of the gas pressure moving an intermediate component out of the way, thus allowing movement of the one or more locking members. In some embodiments, the retaining mechanism comprises a release member arranged to be moved by the gas pressure to allow the one or more locking members to move to the non-engaging position. As described above, the housing may be substantially cylindrical. The releasable module may likewise be substantially cylindrical. In this case the release member may be arranged to be moved axially by the gas pressure, where the axial motion is defined by the length axis i.e. symmetry axis of the cylindrical release member. This may allow the one or more locking members to move radially (i.e. perpendicular to the axis of the cylindrical release member). This movement may be inwards i.e. towards the axis of the release member.

In some embodiments, the fluid container comprises a resilient diaphragm, wherein in the pre-activation configuration the resilient diaphragm is compressed, and wherein after activation, the resilient diaphragm is acted on by the gas pressure to push the release member i.e. the resilient diaphragm is arranged to be movable between a compressed position and a non-compressed, or expanded, position. The resilient diaphragm may comprise a substantially flat portion i.e. a “roof” of the fluid container, and a deformable (e.g. compressible and flexible) webbing portion, which connects the flat portion to the rest of the fluid container. This provides a simple mechanism by which the gas pressure generated in the fluid container may be used to create movement of (at least a part of) the fluid container. Thus, in use, in the pre-activation configuration the resilient diaphragm is compressed, and then after activation, the resilient diaphragm (e.g. the webbing portion) is moved by the gas pressure to a non-compressed, or expanded, position. This movement of the diaphragm can be used to move a component of the retaining mechanism to cause release e.g. the resilient diaphragm may be acted on by the gas pressure to push the release member. The pre-compression of the diaphragm is advantageous since it allows potential energy to be stored within the diaphragm (e.g. the webbing portion), and then the gas pressure can be used to release this stored potential energy of the diaphragm to create a pushing force by the diaphragm. This therefore helps to further minimize the amount of energy required by the trigger mechanism. The fluid container, including the resilient diaphragm, may be made of flexible rubber material, e.g. flexible silicone rubber or other similar elastomer. In some examples, the fluid container (optionally including the resilient diaphragm) may be made of a suitable plastic material, e.g. PETG (polyethylene terephthalate glycol), optionally metallized PETG. In some examples, the resilient diaphragm may be made of a different material to the fluid container, for example a silicone diaphragm in a plastic fluid container.

In some embodiments, the releasable module is arranged to be locally, or internally, activated e.g. the microcontroller may trigger sample collection based on specific signals or specific changes in sensor readings from sensors as discussed below, or from a countdown timer embedded in the controller.

In other embodiments, the releasable module is arranged to be remotely activated e.g. by an external source. In some embodiments the releasable module is arranged to be remotely activated by magnetic field triggering, or through the use of magnetic near-field induction, radio waves, ultrasonic coupling communication, capacitive coupling communication, or galvanic coupling communication. Capacitive coupling provides a particularly simple and low energy communication, thus providing an advantageous trigger mechanism.

In some embodiments the releasable module further comprises a microcontroller (or a system on a chip) and electronic components that enable the possibility of two-way communication between the intestinal capsule assembly and an externally placed transceiver that can relay the communication to the user, and also enables the integration of different sensors. For example, the intestinal capsule assembly, e.g. the releasable module, may comprise at least one sensor e.g. a pH sensor. The microcontroller may be arranged to process data from the at least one sensor and/or transmit this data (or the processed data), to be received by an external transceiver.

In some embodiments, the microcontroller (and optionally other electronic components) are embedded on a flexible circuit board. The flexible circuit board may also embed terminals for contacting battery poles and/or electrically conducting or insulated pads for transmitting and receiving communication signals, and/or sensors, and/or transmitters, and/or electrodes for performing electrolysis. In some embodiments, the flexible circuit board is shaped and/or positioned in the releasable module, so that part of the circuit board (e.g. electrodes) is within the fluid container.

Thus, in use, this part of the circuit board will be immersed in electrolysis solution contained in the fluid container. In some embodiments, a (or another) part of the circuit board is exposed to the exterior of the intestinal capsule assembly e.g. so as to be exposed, in use, to gut fluid. In some embodiments, the parts of the circuit board exposed to the exterior contain pads and components needed for communication and/or sensors, such as a pH sensor e.g. to sense properties of gut fluid, in use. In some embodiments, the controller is programmed to control the flow of electrical currents and signals to and from all parts of the electronic circuit.

In some embodiments the intestinal capsule module may comprise a pull tab arranged to obstruct an electrical flow path from the battery. This helps to prevent the battery draining before use, and the pull tab can be easily removed by a user before final assembly of the intestinal capsule, or even directly before use of the intestinal capsule assembly. In some embodiments, the pull tab comprises at least one electronic connection. This allows charging of the battery and/or re-programming of the microcontroller after final assembly, before the pull tab is removed in order to use the intestinal capsule assembly.

In some embodiments, the intestinal capsule may be packed (i.e. ready for use) within packaging, for example packaging layers, e.g. sterile packaging, e.g. blister packaging. This packaging ensures that the intestinal capsule remains sterile until it is ready to be ingested, and can also help to prevent fluids within the intestinal capsule (e.g., electrolytes, preservatives, lubricants etc.) from drying out.

Thus, there is further provided an intestinal capsule product, comprising an intestinal capsule assembly as described herein above, and a packaging, wherein the intestinal capsule assembly is at least partially retained within the packaging. In some embodiments the intestinal capsule assembly is substantially sealed within the packaging The packaging may be sterile. In some embodiments the packaging may comprise more than one layer, for example with the intestinal capsule assembly arranged between two layers, e.g. for ease of access by peeling the layers apart.

Electrical contacts, which provide an electrical flow path to the battery, may extend beyond the packaging, so that charging and/or programming are possible whilst the capsule assembly remains sealed within the packaging. As mentioned above, in some embodiments the intestinal capsule module comprises a pull tab arranged to obstruct an electrical flow path from the battery, and the pull tab comprises at least one electronic connection. This pull tab (and the electrical connection it comprises) may extend beyond the packaging, such that charging of the battery and/or reprogramming of the microcontroller after final assembly is enabled before the intestinal capsule assembly is removed from the packaging. The applicant has appreciated that this may be a beneficial way of packaging a variety of electronic medical devices, as is described further below.

In some embodiments, the pull tab may be attached to the packaging so that removal of the packaging from the intestinal capsule assembly causes removal of the pull tab and therefore activates the releasable module. In some embodiments, the packaging is arranged such that the pull tab (or at least the electronic connection thereof) extends outside of the packaging, e.g. out of a hole in the packaging or extends out between layers of the packaging.

In some embodiments, the sample chamber comprises, i.e. contains a preservative for preserving certain properties of the sample of intestinal fluid e.g. collected by the sample chamber when the intestinal capsule assembly is in use. This helps to ensure that the desired properties of a sample of intestinal fluid collected within the sample chamber are preserved until a later time at which they are tested. The sample may be in the sample chamber for some time depending on how long the intestinal capsule assembly takes to travel through the intestine of the subject and be expelled.

In some embodiments the assembly comprises a laxative, which is released as a result of release of the retaining mechanism. This may be located e.g. within the housing, or within the releasable module. This laxative is released when release of the retaining mechanism (which happens shortly before sample collection) is triggered. Thus by the time the laxative takes effect there will have been sufficient time for sample collection to take place, and the laxative will then help to reduce the time taken for the intestinal capsule assembly to travel through the intestine and be expelled. This may reduce the time for which the sample is retained within the sample chamber and thereby improve the sample quality.

In some embodiments, a surface of the intestinal capsule assembly (e.g. an outer surface of the housing) is at least partly coated with an ultrasound contrast agent, e.g. to make the device visible during ultrasound examinations. This helps to locate the intestinal capsule assembly using an external ultrasound device, as it travels through the gut of a subject. The ultrasound contrast agent may be a plurality of gas-filled microspheres which reflect ultrasound waves.

In some embodiments, the outer surface of the housing is at least partly coated or draped in a slippery i.e. lubricant, material. Such a material makes the assembly easier to swallow.

The sample chamber defines a volume for collecting a sample of intestinal fluid. In a first set of embodiments of the intestinal capsule assembly according to the first aspect described above, the housing may further comprise a second opening and the sample chamber may be arranged for collecting a sample of intestinal fluid through the second opening. In at least some of these embodiments, the second opening is arranged for intestinal fluid to travel into the sample chamber along a fluid flow path having a direction that is the same as the direction in which the releasable module moves out of the housing through the first opening. For example, intestinal fluid may be drawn into the sample chamber through a second opening at a back end of the assembly while the releasable module moves out of the housing through a first opening at a front end of the assembly.

In some embodiments, the second opening comprises a one-way valve. This helps to ensure that intestinal fluid drawn into the sample chamber does not subsequently leave the chamber before desired e.g. whilst sample collection is still ongoing. In this case, the gas generation means described above may offer a further advantage, specifically to remove any clogs which might be present in the one-way valve and help to force the one-way valve closed.

A further challenge for collection of intestinal fluid is that fluids from the small intestinal lumen are often viscous. Cells lining the intestinal wall continuously produce mucus that helps to hinder microbial pathogens from reaching the intestinal cell wall and which provides a matrix for immobilizing important macromolecules like digestive enzymes, antibodies, and other protectants. Aspirating intestinal fluids is therefore complicated since the mucus will tend to clog the second opening and thereby halt the specimen collection. If the second opening remains clogged, it increases the risk of specimen cross-contamination as the intestinal capsule assembly moves through different parts of the gut. Thus, in addition to the mechanisms described above, which help to move the piston to the stop position, means may additionally, or alternatively, be provided which help to reduce the viscosity of the intestinal fluid as the sample is collected. This will also help to maximise the amount of sample collected and thereby help to move the piston to the stop position.

Thus, in some embodiments the housing comprises at least one filter arranged to cover the second opening, wherein the at least one filter is configured to induce shear in the intestinal fluid as the fluid passes through the filter, so as to reduce the viscosity of the intestinal fluid before the intestinal fluid reaches the second opening. Such a filter creates physical shearing of the intestinal fluids, separating mucins within the fluid which contribute to their viscosity.

The use of a filter upstream of a sample chamber to create or induce shear in intestinal fluid before it is drawn into the sample chamber is considered to be both novel and inventive in its own right, and thus according to a second aspect of the present invention, there is provided an intestinal capsule assembly comprising a sample chamber, for collecting a sample of intestinal fluid, the sample chamber comprising an opening through which the sample is collected, wherein the sample chamber comprises at least one filter arranged to cover the opening, wherein the at least one filter is configured to induce shear in the intestinal fluid as the fluid passes through the filter, so as to reduce the viscosity of the intestinal fluid before the intestinal fluid reaches the opening.

The at least one filter may reduce viscosity of the sample fluid by being arranged such that fluid passing through travels a path including an (approximately) perpendicular change in direction.

In some embodiments, the at least one filter may comprise an outer filter. The outer filter may be configured to induce shear in the intestinal fluid as the fluid passes through the outer filter. The outer filter may be a coarse filter, to prevent any large particles from entering the sample chamber through the (e.g. second) opening. The outer filter may be curved or bent, such that intestinal fluid can be collected from different directions. This curved shape also helps to prevent suctioning of the intestinal capsule assembly onto the side wall of the gut of a subject, since the whole surface of the outer filter does not lie in a plane and therefore only a part of the surface could ever be in contact (e.g. suctioned) to the intestine wall which is substantially planar (relative to the size of the intestinal capsule assembly).

The at least one filter may comprise slots. The slots may be configured to induce shear in the intestinal fluid as the fluid passes through the slots. It may be specifically the outer filter which comprises the slots. The at least one filter may have a substantially circular cross-section, and the slots may be radial with respect to this cross-section.

The slots may be radial with respect to an elongate axis of the housing, which may be cylindrical as mentioned above.

The at least one filter may comprise at least one sharp edge. The sharp edge may be configured to induce shear in the intestinal fluid as the fluid passes over the sharp edge. The skilled person will understand that a filter is considered to be sharp if it is sufficiently sharp to cut through mucins in the intestinal fluid, so as to shear them apart. The at least one sharp edge may be arranged so that fluid passing through the slots then passes over the sharp edge to shear the fluid from the slots.

In some embodiments, additionally or alternatively to the at least one sharp edge, the at least one filter comprises small openings, wherein the openings are misaligned with the slots and/or wherein the openings are smaller than the slots, arranged so that fluid passing through the slots then passes through the openings, to shear the fluid from the slots. The small openings may be configured to induce shear in the intestinal fluid as the fluid passes through the small openings. It will be understood that an opening is “small” if it is of a comparable size to mucins within the intestinal fluid such that the intestinal fluid is squeezed, or stretched, passing through the openings, so as to induce shear, untangling or separating mucins within the intestinal fluid.

The at least one filter may comprise a ridge, an upper edge of the ridge providing the at least one sharp edge, and the openings passing through the ridge. The upper edge of the ridge may be arranged close to the outer filter, leaving only a small gap through which intestinal fluid is able to pass.

In some embodiments the (e.g. second) opening and/or the at least one filter comprises an enzymatic coating which reduces viscosity of the intestinal fluid. In addition, or alternatively, to the physical shearing provided by the one or more filters as described above, this provides enzymatic shearing of the intestinal fluid. The coating may be applied to the edges of the slots and/or the small openings and/or the at least one sharp edge. In some embodiments, the coating contains mucinase.

In a second set of embodiments of the intestinal capsule assembly according to the first aspect described above, the sample chamber may be arranged for collecting a sample of intestinal fluid through the first opening (i.e. the same opening out of which the releasable module moves). In at least some of these embodiments, the first opening is arranged for intestinal fluid to travel into the sample chamber along a fluid flow path having a direction that is opposite to the direction in which the releasable module moves out of the housing through the first opening. For example, intestinal fluid may be drawn into the sample chamber through a first opening at a front end of the assembly while the releasable module also moves out of the housing through the same first opening at the front end of the assembly. In such embodiments of the intestinal capsule, the ejection of the releasable module thus prevents the gut wall from coming into close contact with the first opening that provides a sample inlet, thus minimizing the risk of having the intestinal capsule assembly suctioning onto the side wall of the gut of a subject during sample aspiration.

The arrangement of a sample chamber to release a releasable module, and to draw in fluid through the same opening out of which the releasable module is released, which helps to ensure a safe distance between the gut wall and the sample inlet opening, is considered to be both novel and inventive in its own right. Thus, according to a third aspect of the present invention, there is provided an intestinal capsule assembly, comprising:
a housing, comprising a first opening, and a sample chamber defining a volume for collecting a sample of intestinal fluid along a fluid flow path, wherein the fluid flow path passes through the first opening;
a releasable module; and
a retaining mechanism, for retaining the releasable module within the housing such that the releasable module closes the first opening;
wherein the intestinal capsule assembly has a first, pre-activation configuration in which the releasable module is located within the housing and a second, post-activation configuration, in which the releasable module is located outside of the housing; and
wherein the releasable module is arranged to be activated, so as to cause release of the retaining mechanism, so as to allow movement of the releasable module out of the housing through the first opening, allowing a sample of intestinal fluid to be collected along the fluid flow path.

The first opening may be the only opening formed in the housing. The housing may further comprise a cap, arranged at an end of the housing which is distal from the first opening. The cap may be rounded. This advantageously provides an intestinal capsule assembly which is more easily ingestible due to its shape. The cap may be removable e.g. a screw cap. Such a removable cap can enable convenient access to the inner contents of the intestinal capsule assembly.

As described above, in some embodiments, the intestinal capsule assembly further comprises a piston, wherein the piston defines a wall of the sample chamber and the expansion of the volume of the sample chamber causes movement of the piston towards the first opening.

In some embodiments, the sample chamber may be defined by the housing and the piston. The piston may be arranged to abut (e.g. seal) against an inner wall of the housing. The piston and/or the inner wall of the housing may comprise grooves, or indents, such that the piston does not contact the inner wall around their entire perimeter. This advantageously provides a fluid flow path, passing through the first opening (and then through the groove/indent(s) along which a sample can be drawn into the sample chamber).

The housing may comprise a stop. The stop, which may be a lip around the inner surface of the housing, may be additional, and separate to, a lip around the first opening of the housing, and may be within i.e. not at one end of, the housing. The stop may have a smooth inner edge i.e. be without grooves or indents, such that when the piston reaches the stop, the piston is fully sealed around its perimeter against the stop. This helps to ensure that the sample chamber, defined in part by the piston, remains sealed and conveniently arranged within the housing, which protects the sample chamber.

The piston may comprise at least one tab, arranged to be positioned within a groove or indent of an inner wall of the housing when the piston abuts against the inner wall of the housing. This tab (or tabs) slides in the groove as the piston slides along the length of the housing, and serves to stabilize the piston during sample collection, and also stops the movement of the piston when the tabs contact the lower end of the grooves. Optionally the piston may comprise two tabs e.g. on opposite sides of the piston, this helps to provide a more stabilized and balance force to the piston.

In some embodiments, the piston comprises a one-way valve. This provides a fluid flow path, including the first opening, along which a sample may be drawn into the sample chamber, even where the piston is sealed against an inner wall of the housing along its entire perimeter. The one-way valve also helps to ensure that intestinal fluid drawn into the sample chamber does not subsequently leave the chamber before desired e.g. whilst sample collection is still ongoing. Since the one-way valve provides a fluid flow path (including the first opening) through which a sample may be collected, the inner wall of the housing in such embodiments may be substantially smooth i.e. without indents of projections.

In some embodiments, the releasable module comprises a battery and a fluid container, wherein the releasable module is arranged to be activated, to cause the battery to supply power to the fluid container so as to cause, in use, electrolysis of a fluid within the fluid container to create a gas pressure, wherein the gas pressure causes release of the retaining mechanism, so as to allow movement of the releasable module out of the housing through the first opening. This technique using gas pressure generated by electrolysis to release the retaining mechanism may have any of the further features already described above with reference to the first aspect.

In some embodiments, the housing may comprise at least one filter arranged to cover the one-way valve e.g. the piston may comprise the at least one filter, wherein the at least one filter is configured to induce shear in the intestinal fluid as the fluid passes through the filter. This filter may have any of the features of the filter described above (and as disclosed with reference to the second aspect).

In some embodiments according to any of the aspects disclosed above, the intestinal capsule assembly may comprise a dissolvable blocking member, arranged to prevent movement of the piston to a position in which the sample chamber is sealed i.e. to prevent the piston from moving to a position in which the first opening is closed. For example, the dissolvable blocking member may be arranged in a groove of the inner wall of the housing. After some time of exposure to gut fluids entering the sample chamber along the fluid flow path the blocking member will be dissolved by these fluids, and closing of the sample chamber will be possible. This helps to increase the length of sample collection time.

In some embodiments according to any of the aspects disclosed above, an external edge of the piston (e.g. on the face which does not define the sample chamber or otherwise facing outwardly from the first opening) comprises a sharp edge. This sharp edge will shear viscous materials as the piston moves into the maximum, or end position, e.g. contacting the lip, and therefore helps to ensure proper sealing of the sample chamber or sealing of the piston against the housing in its end position.

In some embodiments according to any of the aspects disclosed above, the first opening and/or the grooves and/or the one-way valve comprises an enzymatic coating which reduces viscosity of the intestinal fluid. This provides enzymatic shearing of the intestinal fluid. In some embodiments, the coating contains mucinase.

According to a fourth aspect of the present invention, there is provided a product comprising an electronic medical device, and a sterile packaging, at least partly enclosing the electronic medical device, the electronic medical device comprising:
a battery; and
at least one electronic connection;
wherein the packaging is arranged such that the at least one electronic connection extends outside of the packaging, e.g. out of a hole in the packaging or passing out between layers of the packaging.

While the at least one electronic connection extends outside of the packaging, the packaging may still be substantially sterile, for example by sealing around the electronic connection at its point of exit. Thus, in some embodiments, the sterile packaging may fully enclose the electronic medical device, for example sealing the electronic medical device inside the packaging.

The electronic medical device may further comprise a pull tab, wherein the pull tab comprises the at least one electronic connection. The pull tab may be arranged to obstruct an electrical flow path from the battery. The pull tab may be attached to the packaging so that removal of the electronic medical device from the packaging causes removal of the pull tab and therefore activates the electronic medical device.

The electronic medical device may be an intestinal capsule assembly having any of the features laid out above.

It will be understood that all of the features described above with respect to the intestinal capsule assembly according to the first aspect of the invention, may likewise be present in an intestinal capsule assembly according to the second aspect and the third aspect. Similarly the filter features described with respect to the second aspect may likewise be present in an intestinal capsule assembly according to the first aspect and the third aspect.

Features of any aspect or embodiment described herein may, wherever appropriate, be applied to any other aspect or embodiment described herein. Where reference is made to different embodiments or sets of embodiments, it should be understood that these are not necessarily distinct but may overlap.

### BRIEF DESCRIPTION OF THE DRAWINGS

Certain preferred embodiments of the invention will now be described, by way of example only, with reference to the accompanying drawings, in which:

FIG. 1 is a schematic drawing showing a side view of an intestinal capsule assembly according a first embodiment of the present invention, shown in the pre-activation configuration;

FIG. 2 is a cross-sectional side view showing the intestinal capsule assembly of FIG. 1 in greater detail;

FIG. 3 is a perspective cross-sectional view of the assembly of FIG. 2, shown viewed slightly from below;

FIG. 4 is a perspective cross-sectional view of the assembly of FIG. 2, shown viewed slightly from above;

FIG. 5 is a cross-sectional view showing a segment of the at least one filter of the sample chamber of FIG. 2;

FIG. 6 is a view of the filter of FIG. 5, illustrating two possible fluid paths through the filter;

FIG. 7 is a cross-sectional side view of the assembly of FIG. 2, showing the assembly as release of the retaining mechanism is triggered;

FIG. 8 is a cross-sectional side view of the assembly of FIG. 2, showing the assembly in a position in which the retaining member has just been released;

FIG. 9 is a cross-sectional side view of the assembly of FIG. 2, showing the releasable module moving out of the sample chamber through the first opening;

FIG. 10 is a cross-sectional side view of the assembly of FIG. 2, showing the releasable module fully outside the sample chamber, with the piston moving through the sample chamber towards the first opening;

FIG. 11 is a schematic side view showing four stages of movement between the pre-activation configuration and the post-activation configuration, with the assembly within a small intestine;

FIG. 12 is a cross-sectional side view of the assembly of FIG. 2, showing the assembly with the releasable module fully outside the sample chamber in the post-activation configuration, with the piston sealing the first opening;

FIG. 13 is an exploded view showing the various components of the releasable module;

FIG. 14 is an exploded view showing the various components of the intestinal capsule assembly according to a first embodiment of the invention;

FIG. 15 is a schematic drawing showing a side view of an intestinal capsule assembly according a second embodiment of the present invention, shown in the pre-activation configuration;

FIG. 16 is a schematic drawing showing a side view of an intestinal capsule assembly according a second embodiment of the present invention, shown between the pre-activation and post-activation configurations, showing the fluid flow path;

FIG. 17 is a cross-sectional side view showing the intestinal capsule assembly of FIGS. 15 and 16 in greater detail;

FIG. 18 is a perspective cross-sectional view of the assembly of FIG. 17, shown viewed slightly from below;

FIG. 19 is a perspective cross-sectional view of the assembly of FIG. 17, shown viewed slightly from above;

FIG. 20 is a cross-sectional side view of the assembly of FIG. 17, showing the releasable module moving out of the sample chamber through the first opening, and showing the fluid flow path;

FIG. 21 is a perspective cross-sectional view of the housing of the assembly of FIG. 17, shown viewed slightly from below, with a number of the components omitted to show more clearly the grooves of the inner surface of the housing;

FIG. 22 is a cross-sectional side view showing the housing of FIG. 21;

FIG. 23a is a cross-sectional view of the housing of FIG. 22, taken along the line AA as shown in FIG. 22;

FIG. 23b is a cross-sectional side view of the housing of FIG. 22;

FIG. 24 is a perspective cross-sectional view of an intestinal capsule assembly according to a third embodiment, shown viewed slightly from below, with a number of the components omitted to show more clearly one-way valve;

FIG. 25 is a perspective cross-sectional view of the housing of FIG. 24, shown viewed slightly from above;

FIG. 26 is a cross-sectional side view showing the housing of FIG. 24;

FIG. 27 is a cross-sectional side view of the assembly of FIG. 17, showing the assembly with the releasable module fully outside the sample chamber in the post-activation configuration, with the piston sealing the first opening;

FIG. 28 is an exploded view showing the various components of the intestinal capsule assembly according to the second embodiment of the invention;

FIG. 29 is a side view showing the stages of sample collection using a capsule assembly according to the second or third embodiment of the present invention;

FIG. 30 is a perspective view showing an alternative retaining mechanism;

FIG. 31 is a perspective view showing the surround of the retaining mechanism of FIG. 30;

FIG. 32 is a perspective view showing the release member of the retaining mechanism of FIG. 30;

FIG. 33 is a cross-sectional side view showing operation of the retaining mechanism of FIG. 30;

FIG. 34 is a cross-sectional side view showing operation of the retaining mechanism of FIG. 30;

FIG. 35 is a cross-sectional side view showing operation of the retaining mechanism of FIG. 30; and

FIG. 36 is a cross-sectional view showing an intestinal capsule product, comprising an intestinal capsule assembly at least partially retained within a packaging.

### DETAILED DESCRIPTION

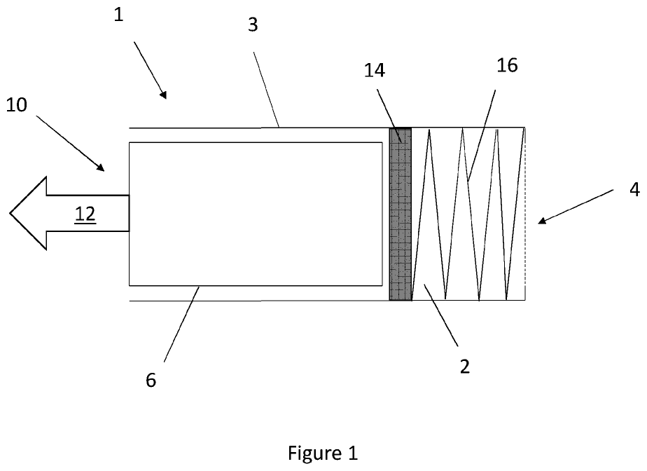

FIG. 1 shows an intestinal capsule assembly 1 according to a first embodiment of the present invention. The intestinal capsule assembly 1 includes a housing 3. The housing 3 has a first opening 10 and a second opening 4. The housing 3 includes a sample chamber 2, defined or bounded by the sides of the housing 3, the second opening 4 and a piston 14. In use, a sample of intestinal fluid is collected through the second opening 4 into the sample chamber 2.

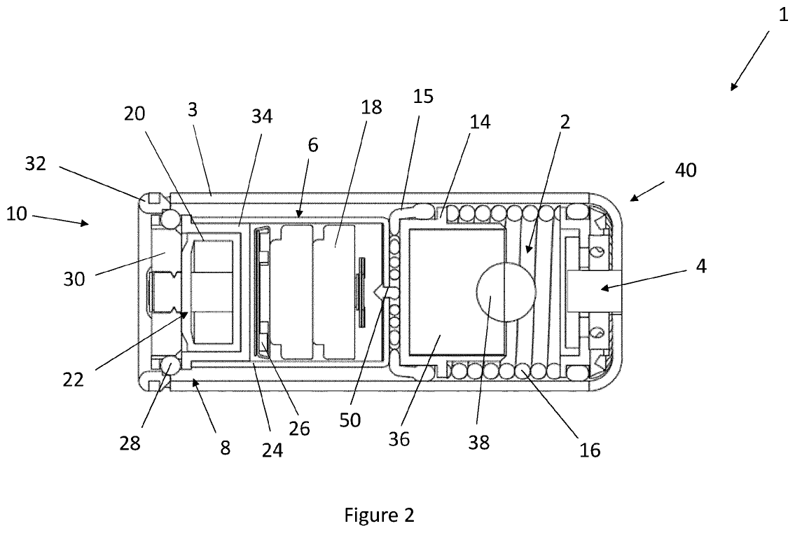

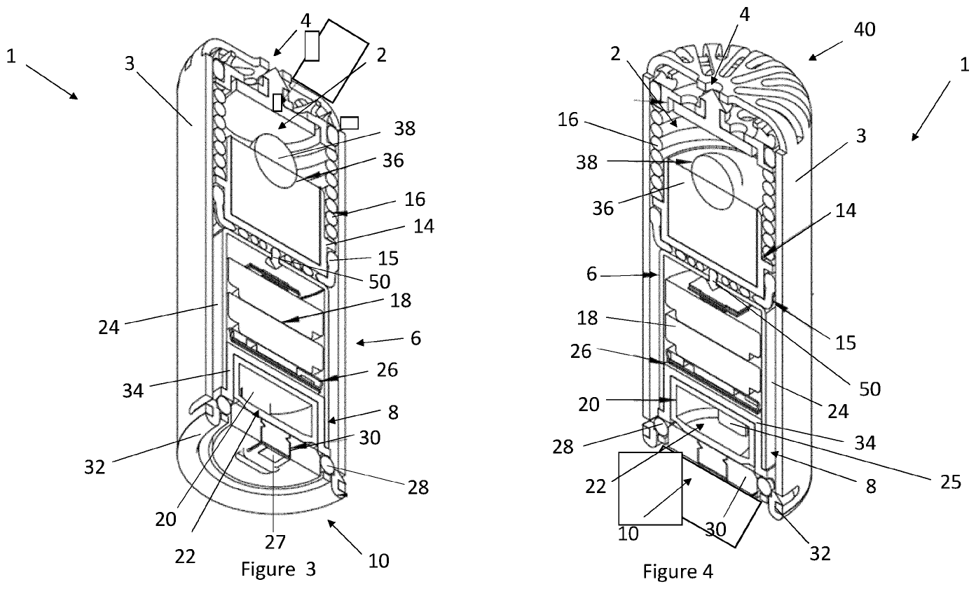

A releasable module 6 is located within the housing 3 and retained there by a retaining mechanism 8 seen in FIGS. 2 and 3 (and also seen in FIGS. 4, 7, 8, 9, 10 and 12). In use, the releasable module 6 exits the housing 3 through the first opening 10 once released by the retaining mechanism 8, this movement being represented by arrow 12. The intestinal capsule assembly 1 is shown in FIG. 1 in a first, pre-activation configuration in which the releasable module 6 is located within the housing 3.

The sample chamber includes the piston 14 and a spring 16, which is biased between the housing 3 and the piston 14. The spring 16 is biased i.e. compressed, and therefore stores potential energy, and will therefore expand once the forces holding it in the compressed state are removed. Once the retaining mechanism 8 is released, allowing movement of the releasable module 6, the spring 16 begins to expand, applying a force to the piston 14 which in turn pushes the releasable module 8 through the first opening 10, out of the sample chamber 2. The movement of the piston 14 through the housing 3 also expands the volume of the sample chamber 2, since one side of the sample chamber 2 is defined by the piston 14.

The intestinal capsule assembly 1 is shown in greater detail, again in the pre-activation configuration, in FIGS. 2, 3 and 4.

As can be seen in these Figures, the releasable module 6, includes a battery 18 (made up of two individual cells) and a fluid container 20 contained within a housing 24. In this example, the fluid container 20 is made of flexible silicone rubber. However, it may be made of other suitable material(s), as described above. The fluid container includes a diaphragm 22, which in the configuration of FIG. 2 is in a compressed state, giving the fluid container 20 a rectangular cross-section. The functioning of the diaphragm 22 will be described further below.

The releasable module 6 further contains electronic circuitry 26. The electronic circuitry 26 enables wireless two-way communication with an operator computer via an externally placed signal transceiver. This allows release of the retaining mechanism 8 to be triggered remotely, as described below, by activating the battery 18. The intestinal capsule assembly may include sensors (not shown) for example on a signal or sensor pad 27, fixed to the circuit board, as shown in FIG. 3, for example a pH sensor. Data from these sensors can also be transmitted using the electronic circuitry.

The releasable module 6 also includes the retaining mechanism 8, seen in cross-section in FIGS. 2, 3 and 4. The retaining mechanism 8 shown in this particular example includes four ball locks 28 (only two are seen in cross-section), a release member 30 and a surround 34. The ball locks 28 are arranged in frictional engagement between a lip 32 formed around the first opening 10 and the release member 30. The pre-compression of the diaphragm 22 provides some additional push force bias against the release member 30, further reducing the gas pressure needed for release.

The release member 30 is arranged to be moved by the diaphragm 22, which is moved by the gas pressure generated in the fluid container 20. This moves the release member 30 axially away from the ball locks 28 and the surround 34, as described below, creating a space into which the ball locks 28 are able to move inwards to the non-engaging position so they no longer engage with the lip 32, allowing the releasable module 6 to move out of the first opening 10. This retaining mechanism 8 provides an energy-efficient mechanism, capable of controlling the strong compression spring 16 needed for aspirating viscous fluid into the sample chamber 2.

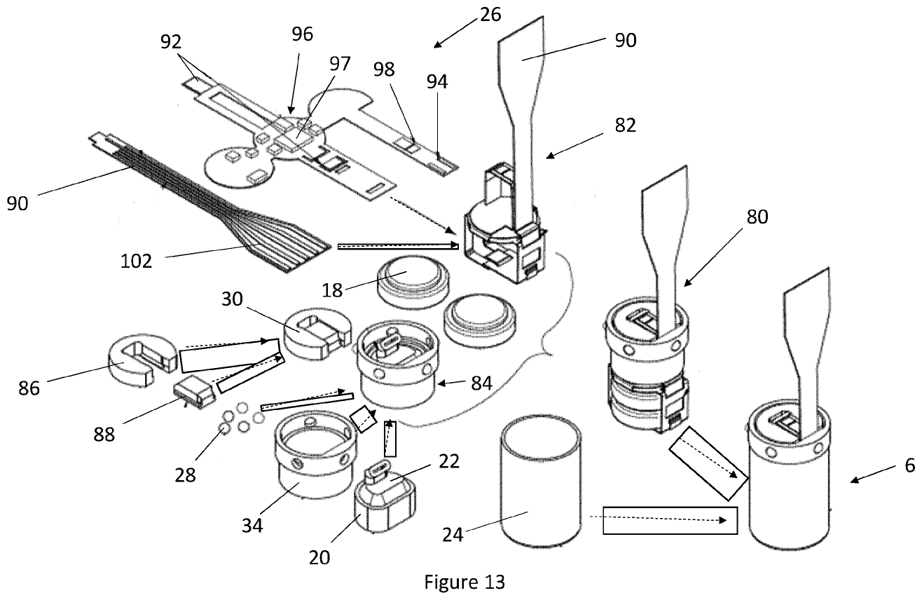

The release of the retaining mechanism 8, used to release the releasable module 6 and thereby allow the spring-loaded piston 14 to move, is powered by gas pressure that is generated in the fluid container 20 by electrolysis. The electrolysis solution is based on buffered formic acid. The fluid container 20 contains platinum-based catalyst-coated electrodes 94 (seen in FIG. 13 but not shown in FIG. 4, since they are arranged on the other side of a part 25 of the circuit board shown) which are powered by the battery 18, and contact the electrolysis solution within the fluid container 20. The battery 18 selectively powers the electrodes under the control of the microcontroller 97 in the electronic circuitry 26. When exposed to a small amount of current and voltage through the platinum-based catalyst-coated electrodes, the electrolysis solution generates considerable gas pressure from the decomposition of formic acid into carbon dioxide and hydrogen gas.

Once the releasable module 6 is released, the spring 16 can begin to expand, moving the piston 14 towards the first opening 10 and drawing a sample in through the second opening 4. One of the challenges when collecting the sample is that intestinal fluid is often very viscous and therefore difficult to aspirate.

To assist with this problem, the housing 3 further includes a filter 40, arranged over the second opening 4, such that intestinal fluid passes through the filter 40 before reaching the second opening 4. The filter 40 is seen in more detail in FIG. 5. The filter includes a coarse outer filter 42 which includes radial slots 43, and an inner ridge 44. The outer filter 42 is curved, having a total curve of approximately 90 degrees, such that the surface of different parts of the outer filter 42 are substantially perpendicular to each other. The slots 43 extend along the curved surface such that they extend down the side of the outer filter 42. The inner ridge 44 has a sharp upper edge 46, which is close to a lower surface of the outer filter 42. The inner ridge also includes small openings 48, which pass from one side of the ridge 44 to the other. The small openings 48 do not align directly with the outer filter slots 43 i.e. they are misaligned.

The sharp upper edge 46 and small openings 48 of the ridge 44 are coated with mucin-degrading enzymes (i.e. mucinases) that enzymatically break down mucins, which are the large proteins that are mainly responsible for making gastric juices viscous.

Below the filter 40 (i.e. further along the sample flow path) is the second opening 4, which is a flexible one-way valve. The sample is drawn into the sample chamber 2.

The sample chamber contains preservatives 36, and gas generation means 38, which will be discussed in greater detail below with reference to the later Figures.

An O-ring 15 surrounds the piston 14, forming a seal against the housing 3. The O-ring is shaped to contact the lip 32 when the piston 14 moves to its maximum movement position within the housing 3, the stop position, to help seal the sample chamber 2.

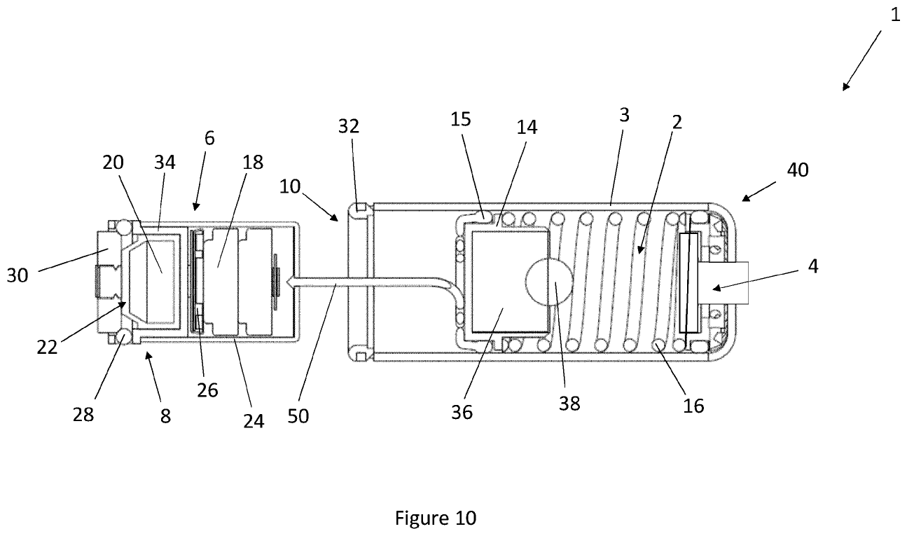

A flexible attachment 50 made of silicone rubber connects the piston 14 to the releasable module 6, via the O-ring 15, as seen in FIG. 10. This flexible attachment 50 provides a system for harvesting energy from intestinal contractions, or peristalsis, to help generate the vacuum needed to successfully aspirate viscous fluids, as described below. The flexible attachment 50 is designed to withstand a certain amount of pulling forces before breaking. Even if the flexible attachment 50 is broken during movement of the intestinal capsule assembly 1 through the intestine, the housing 3, containing the sample chamber 2, and the releasable module 6, can simply be retrieved separately.

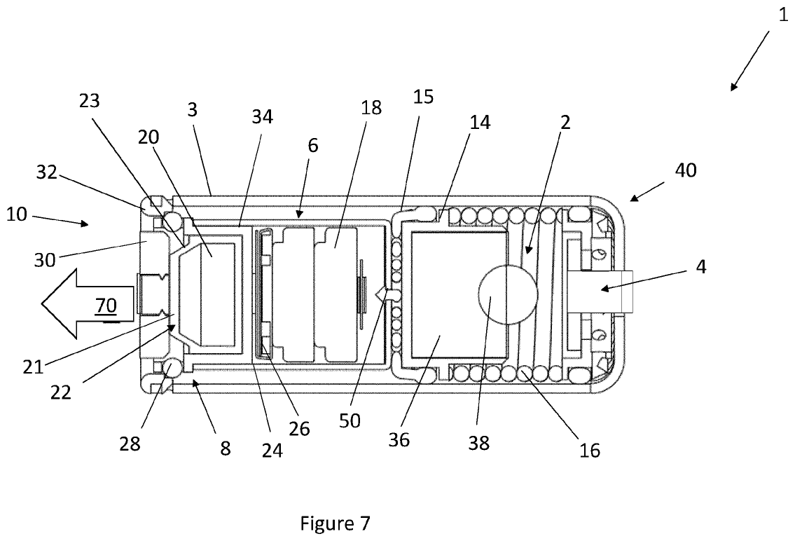

The functioning of the intestinal capsule assembly 1 as it moves from the pre-activation configuration (shown in FIG. 2) to the post-activation configuration will now be described with reference to FIGS. 7-12.

Release of the retaining mechanism is activated by the microcontroller 97 in the electronic circuitry 26. This may be based on signals received through capacitive coupling. Once the microcontroller 97 in electronic circuitry 26 receives the activation signal, the microcontroller 97 channels electrical current from the battery 18 to a fluid within the fluid container 20, via electrodes discussed above so as to cause electrolysis of the fluid. Electrolysis of the fluid creates a gas, which increases the pressure inside the fluid container 20, which is (at least substantially) sealed.

As seen in FIG. 7, the diaphragm 22 includes a flat portion 21, and a deformable webbing portion 23, which is flexible and compressible. The increase in pressure inside the fluid container 20 causes the webbing portion 23 of the diaphragm 22 of fluid container 20 to be pushed. As a result the webbing portion 23 expands, allowing the roof portion 21 to move from its pre-compressed position, giving the rectangular cross-section seen in FIG. 2, to the shape shown in FIG. 7, in which the webbing portion 23 expands, so that the diaphragm 22 has a trapezoid shape in addition to the rectangular non-compressed shape. This expansion is along the elongate axis of the releasable module 6, which is cylindrical i.e. the expansion is axial.

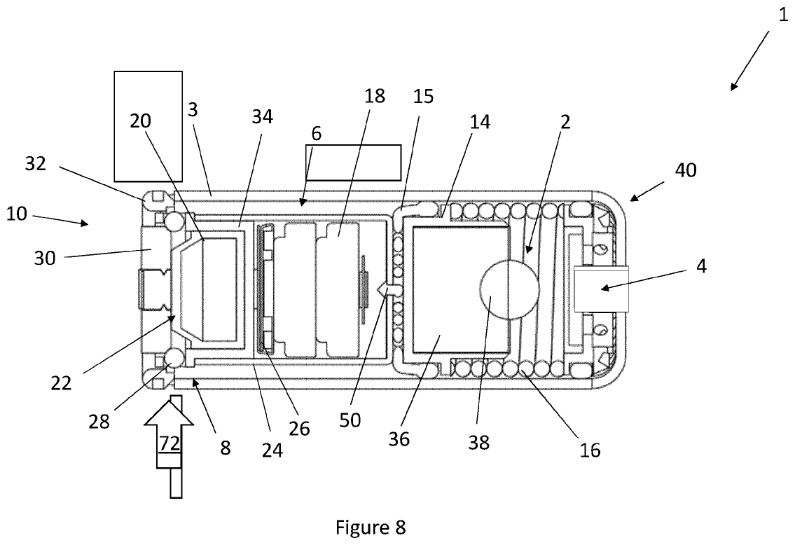

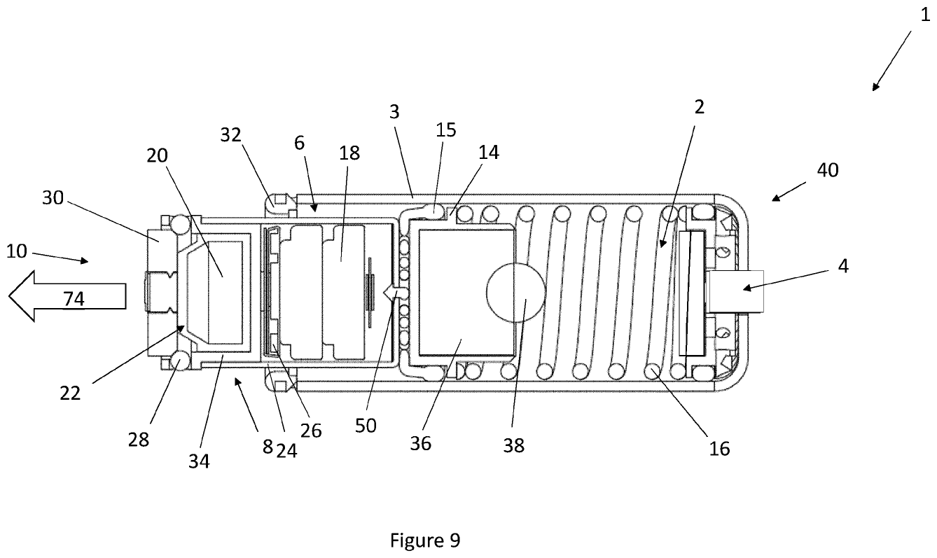

The movement of the diaphragm pushes the release member 30 along the axial direction, away from the rest of the releasable module 6, as represented by the arrow 70 in FIG. 7. This creates a separation between the release member 30 and the surround 34, close to the housing 24 of the releasable module 6. This separation is sufficiently large for the ball locks 28 to move radially inwards, towards the axis of the releasable module 6, into the separation. This motion is represented by arrow 72 in FIG. 8. As a result of this movement the ball locks 28 are no longer frictionally engaged with the lip 32, and the releasable module 6 is free to start moving out of the housing 3 through the first opening 10, as shown in FIG. 9.

The spring 16 begins to expand, moving the piston 14 and O-ring 15 through the housing 3 towards the first opening 10. This pushes on the releasable module 6, which is between the piston 14 and the first opening 10, moving it axially through the first opening 10 as represented by arrow 74 in FIG. 9.

There may be a laxative arranged somewhere within the housing e.g. between the releasable module 6 and the O-ring 15, such that when release of the releasable module 6 is triggered, possibly at the point where the releasable module 6 fully exits the housing, the laxative is released into a subject's intestine. This will help to speed up the movement of the intestinal capsule assembly through the gut after a specimen has been collected and, thereby, speed up recovery.

As the releasable module 6 starts to move towards the first opening 10, the movement of the piston 14 creates a pressure differential, with low pressure inside the sample chamber 2. This pressure causes the one-way valve 4 to open, and intestinal fluid begins to be drawn into the sample chamber 2.

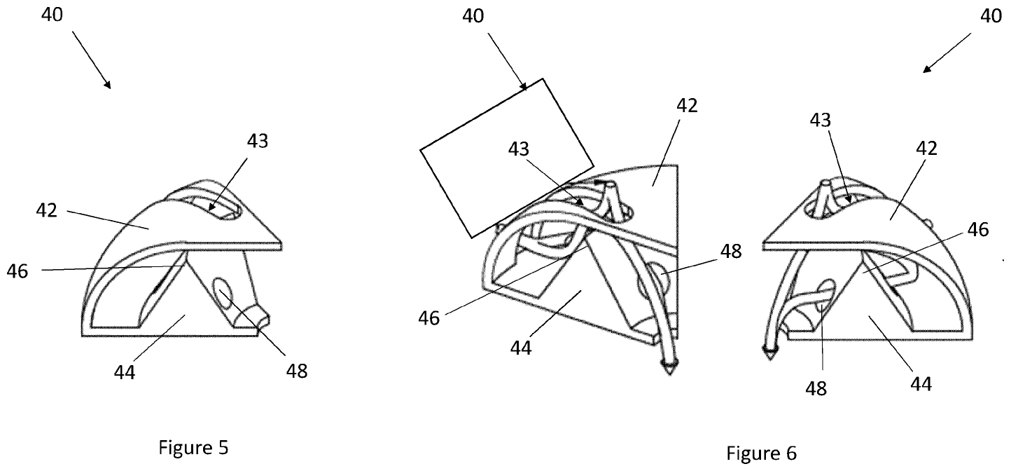

As the intestinal fluid is drawn into the one-way valve 4, it must first pass through filter 40. The possible paths of intestinal fluid through the filter 40 are shown in FIG. 6. As shown in the left hand view, fluid entering through the side of slot 43 or from above the slot 43 may pass over the sharp edge 46. Alternatively, fluid entering the slot 43 from above, or from the side, may pass through small opening 48, as shown on the right hand side of FIG. 6.

These two layers of filters 42, 44 provide a large inlet surface area, as well as a large number of sharp edges that will shear and help to reduce specimen viscosity before it reaches the one-way valve 4. The filters 42, 44 are arranged such that fluid passing through them travels a path including an approximately perpendicular change in direction. The intestinal fluids to be collected are often viscous due to the presence of long chains of glycosylated proteins called mucins. By forcing this fluid though narrow openings over a large surface area, and guiding it through winding, narrow passages and across sharp corners and edges, some of the mucins are stretched and untangled to make the fluids less viscous and, thereby, reduce the risk of clogging the device inlet. Thus by making the channels in these two filters perpendicular to each other, by introducing a small gap between the filters, and by ensuring that the edges of the filter openings are sharp edges, these filters will mechanically stretch and shear mucoid fluids, reducing viscosity.

The outer filter 43 curves around the capsule corners so that fluids can be aspirated i.e. drawn in, across two different faces. This helps to ensure that the capsule assembly 1 does not suction itself to the intestinal wall or to food particles present in the gut during aspiration.

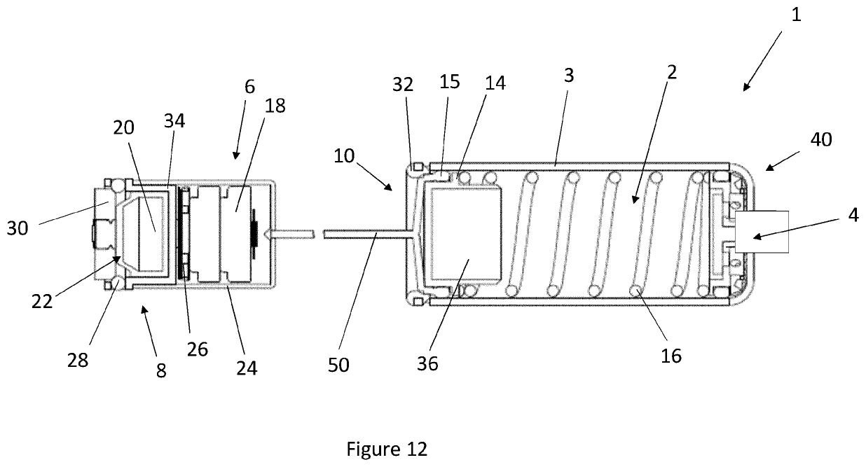

The releasable module 6 will continue to move until it is completely outside of the housing 3, in the post-activation configuration, as shown in FIG. 10. As seen in FIG. 10, full ejection of the releasable module 6 may occur without the piston 14 moving all the way through the housing towards the first opening 10. The position, referred to as the stop position, is the position in which the piston 14 has travelled through the housing as close as possible to the first opening 10, at which point the housing 3 (specifically the lip 32) stops the piston from exiting through the first opening 10. FIG. 12 shows the stop 14 (and O-ring 15) in the stop position, sealing the first opening 10.

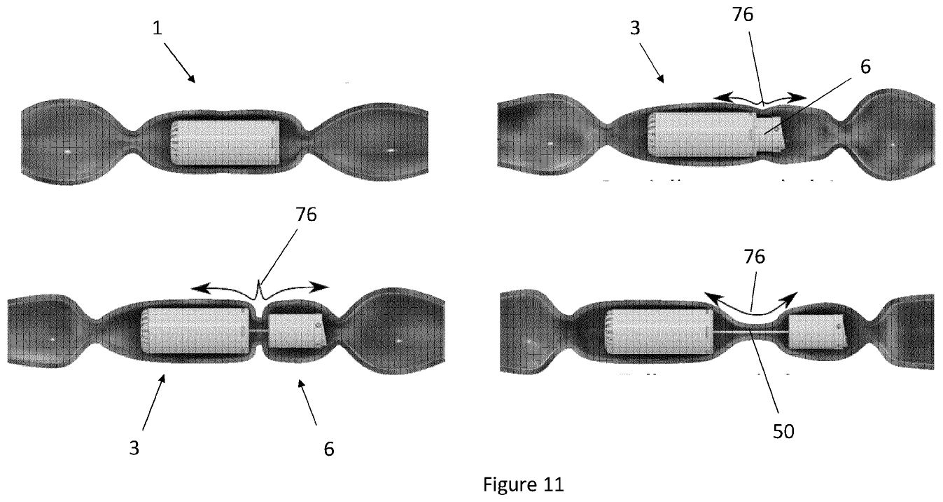

The vacuum created by the piston 14 will decrease as the compression spring 16 continues to expand, and thus may not be sufficient to push the piston all the way to the stop position. To compensate for lower compression spring forces the flexible attachment 50 is used to harvest peristalsis, pulling the piston 14 towards the stop position. The attachment means 50 can be seen in FIG. 10, and will be explained with reference to FIG. 11.

FIG. 11 shows four stages as the intestinal capsule assembly 1 moves through an intestine. In the upper-left image the intestinal capsule assembly 1 is in the pre-activation configuration. In the upper right image, the releasable module 6 has passed partly out of the housing 3, as shown in FIG. 9. In the lower left image, the releasable module 6 has now fully exited the housing 3, as shown in FIG. 10. The action of peristalsis is represented by arrow 76. Repeated contractions and relaxations moving along the length of the intestine push both the releasable module 6 and the housing 3 down the length of the intestine. However, each is acted on separately at slightly different times, thus at certain times they are pulled away from each other, in opposite directions. This creates tension in the flexible attachment 50 which pulls on the piston 14, moving it through the housing 3, towards the stop position, until the piston 14 is in the fully extended, stop position, as shown in the lower right image.

If, even after the action of the spring 16 and the flexible attachment 50, the piston 14 has not moved all the way to the stop position (seen in FIG. 12), it may further be pushed into the stop position by pressure created in the sample chamber 2 by gas created by the gas generation means 38.

The gas generation means 38 are gas generation beads, with a water-soluble polysaccharide coating. After the sample of intestinal fluid has been in the sample chamber 2 for a short period of time e.g. a few minutes, (partially) submerging the gas generation means 38, the intestinal fluid will degrade the outer coating on the gas generation means 38. Then water in the intestinal fluid reacts with the gas generating chemicals in the gas generation means 38, generating gas, causing an increase of pressure in the sample chamber 2. This pressure pushes the piston 14 towards the stop position, helping the O-ring 15 to seal against the lip 32, and also helping to seal the one-way valve 14. The resulting slightly positive pressure inside the sample chamber 2 will reduce the risk of additional fluids entering the sample chamber 2 as the housing 3 continues to travel through the gut, thereby reducing the risk of cross-contamination.

Once the piston 14 moves to the stop position the O-ring 15 is sealed against the inner surface of the housing and also at least partially sealed against the lip 32 at the first opening 10. If there is pressure within the sample chamber 2 this will help to seal the O-ring 15 against the lip 32, and the pressure also seals the one-way valve 14.

During the aspiration process, and once the sample chamber is sealed, the collected fluid sample dissolves the preservatives 36 within the sample chamber 2. These help to stabilize the analytes of interest in the fluid sample until the sample chamber 2 (and therefore its contents) can be retrieved.

FIG. 13 is an exploded perspective view showing the components of the releasable module 6. As is represented by the dashed arrows, the releasable module 6 is made up of the module housing 24, and an assembly 80 including a number of inner components. As represented by the bracket, the assembly 80 is made up of an electronic assembly 82, battery 18, retaining assembly 84 and release member 30 (which together form the retaining mechanism 8).

As shown, the release member 30 includes a plate 86, and a plug 88, as represented by the dashed arrows. The plug 88 slides or clips into a fitted connection with plate 86, and is arranged, in use, over the fluid container 20, specifically the diaphragm 22, such that the plug 88 seals the fluid chamber 20 and also so that the release member 30 is moved axially (i.e. lifted, in the view shown) when the diaphragm 22 de-compresses.

The retaining assembly 84, which together with the release member 30 forms the retaining mechanism 8, is made up of ball locks 28, surround 34 and fluid container 20, as represented by the dashed arrows. The ball locks 28 fit into circular openings provided in an upper lip edge of the surround 34, which help to keep them in place. This example shows five ball locks 28, although in the cross-sections of the earlier Figures, four are shown for convenience. Any suitable number may be used, as long as they are sufficient to resist the force of the spring 16. Diaphragm 22 is shown uncompressed where it is shown alone (below the dashed arrow). Where assembled inside the retaining assembly 84, the diaphragm is shown in the compressed state.

The electronic assembly 82 includes electronic circuitry 26, and a pull-tab 90. The electronic circuitry 26 includes battery connectors 92, arranged to be connected to batteries 18 and electrodes 94, arranged to contact electrolysis solution within the fluid container 20, as described above. The electronic circuitry 26 further includes communication components 96 and a microcontroller 97, able to communicate via capacitive coupling or by other wireless means with an external device. This allows an operator to remotely signal the communication components 96 and microcontroller 97, and this will cause them to activate or enable a connection between the battery connectors 92 and the electrodes 94. When assembled into the releasable module 6, this activation causes electricity to be supplied to the electrolysis solution within the fluid container 20, causing gas production and triggering release of the releasable module 6 as described above.

The electronic circuitry 26 also includes sensors 98, which may include, for example, a pH sensor e.g. as part of the signal/sensor pad 27, shown in FIG. 3. The electronic circuitry 26 may include at least one antenna. Data from the sensors 98 can be transmitted to an external operator using the communication components 96 and microcontroller 97.

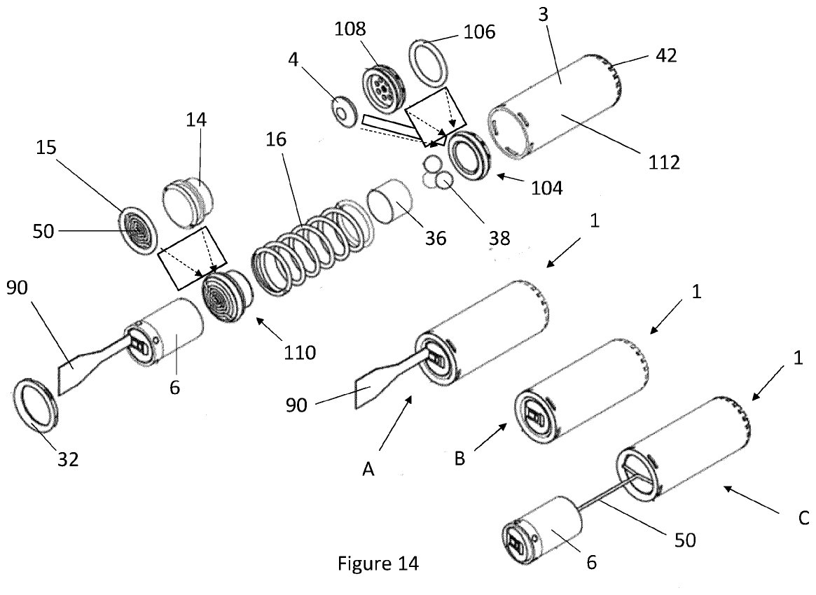

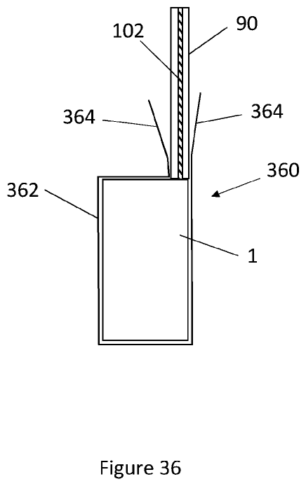

The pull-tab 90 is arranged with the electronic circuitry 26 to form the electronic assembly 82. The pull tab 90 is arranged to form a physical barrier between the battery 18 and the electronic circuitry 26, so that nothing is electrically connected to the battery 18. This blocks battery drainage. The pull tab also includes electrical connections 102, which enable re-programming of the electronic circuitry 26 and charging of the battery 18 after the intestinal capsule assembly 1 has been fully assembled, as shown in FIG. 14. By pulling the tab, the electronic circuitry 26 is made active (i.e. the electronic circuitry 26 is live; this is separate to activation or triggering of the release mechanism) and the capsule can be ingested.

Where the intestinal capsule assembly 1 is placed within a packaging, the pull tab 90 (including the electrical connections 102) can be arranged to extend outside of the packaging, as shown in cross-section in FIG. 36. FIG. 36 shows an intestinal capsule assembly 1 sealed within outer sterile packaging 362 (such as a blister pack), to give a resulting intestinal capsule product 360. The packaging 362 ensures that the intestinal capsule assembly 1 remains sterile until it is ready to be ingested, and also helps to prevent fluids within the intestinal capsule (e.g., electrolytes, preservatives, lubricants etc.) from drying out. The pull tab 90 extends outside the packaging 362 from between layers 364 of the packaging. The pull tab 90 is attached to the packaging 362 by adhesive so that removal of the packaging 362 from the intestinal capsule assembly 1 causes removal of the pull tab 90 and therefore activates the intestinal capsule assembly 1.

The electronic circuitry 26 is conveniently arranged on a flat printed circuit board, which is arranged to be folded to give the three-dimensional shape of the electronic assembly 82 configuration, which includes an opening sized to receive the battery 18 (made up of two individual cells).

As shown in the exploded view of FIG. 14, this releasable module 6, still connected to pull tab 90, is then assembled together with other components within the housing 3 to give the intestinal capsule assembly 1. Although not visible, the outer surface of the housing 3 is coated with a plurality of gas-filled microspheres 112 which reflect ultrasound waves, providing an ultrasound contrast agent to make the device visible during ultrasound examinations.

The components are assembled inside housing 3, which includes the outer filter 42. Beneath the filter 42, within the housing 3, there is arranged a sealing assembly 104. As represented by the dashed arrows, the sealing assembly 104 includes a O-ring 106, arranged to form a seal against the housing 3, a lower filter 108, arranged to include the ridges 44 discussed above, and a one-way valve 4, providing the second opening into the sample chamber.

The sample chamber is defined at one end by the sealing assembly 104, along its length by the inner wall of the housing 3, and at its other end by a piston assembly 110. The piston assembly 110 includes the piston 14, the O-ring 15, which seals against the inner wall of the housing 3, and the flexible attachment 50, attached to the piston 14 via the O-ring 15.

The spring 16 is biased between the sealing assembly 104, and the piston assembly 110. Inside the coils of the spring 16 (and therefore within the sample chamber 2 which is defined in part by the inner walls of the housing 3, the preservative 36 and the gas generating beads 38 are arranged.

Once these components are all positioned within the housing, the releasable module 6 is pushed against the surface of the piston assembly 110 to which it is adjacent, pushing the releasable module 6 inside the housing 3 and in doing so compressing the spring 16 to the biased position. Once the releasable module 6 is entirely within the housing 3, lip 32 is clipped or snapped onto the end of the housing 3, engaging with the ball locks 28 of the releasable module 6 and preventing the releasable module 6 from sliding back out of the housing 3 (until release).

FIG. 14 then shows the assembled intestinal capsule assembly 1, first in the inactivated condition, labelled as A, in which the pull tab 90 is preventing battery connection. In the pre-activation condition, labelled as B, in which the pull tab 90 has been removed, so the intestinal capsule assembly 1 is ready to be swallowed by a subject. FIG. 14 further shows the intestinal capsule assembly in the post-activation condition, labelled as C, in which the releasable module 6 has fully exited the housing.

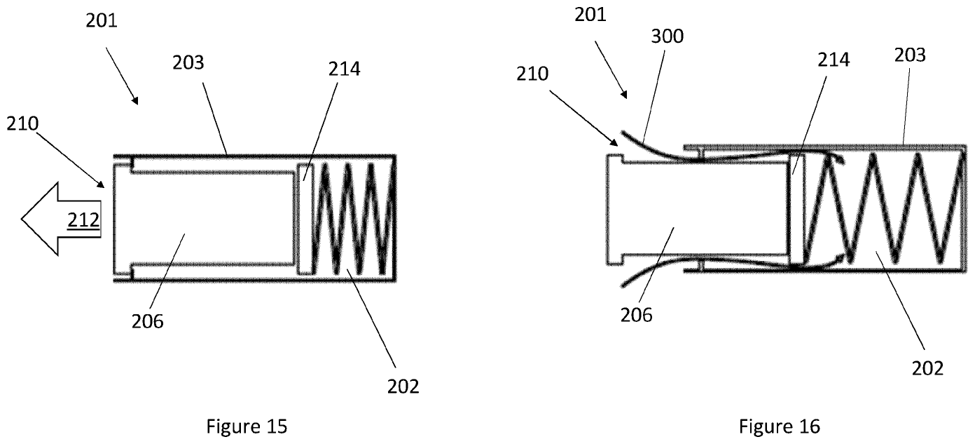

FIG. 15 is a schematic drawing showing a side view of an intestinal capsule assembly according a second embodiment of the present invention. This intestinal capsule assembly 201 has many features in common with the first embodiment, particularly the features of the release mechanism, piston and spring. Like reference numerals will be used throughout for corresponding features (with the value increased by 200 for the second embodiment, compared to the reference numerals used for the first embodiment). Thus, the intestinal capsule assembly 201 includes a housing 203. The housing 203 has a first opening 210. The housing 203 includes a sample chamber 202, defined or bounded by the sides of the housing 203, and a piston 214.

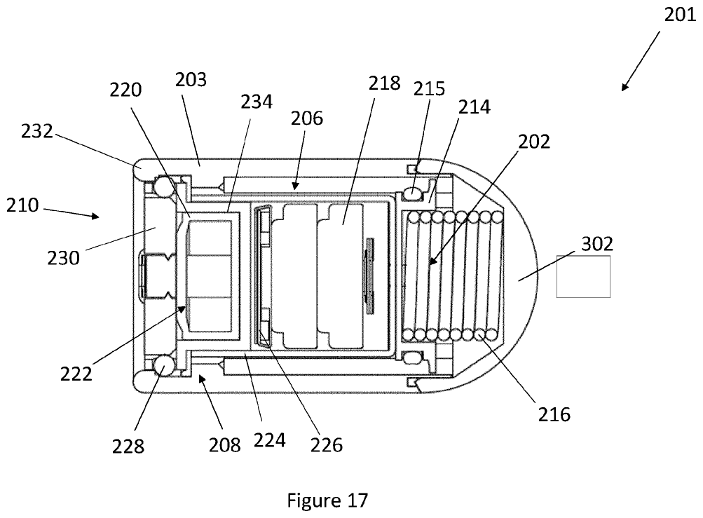

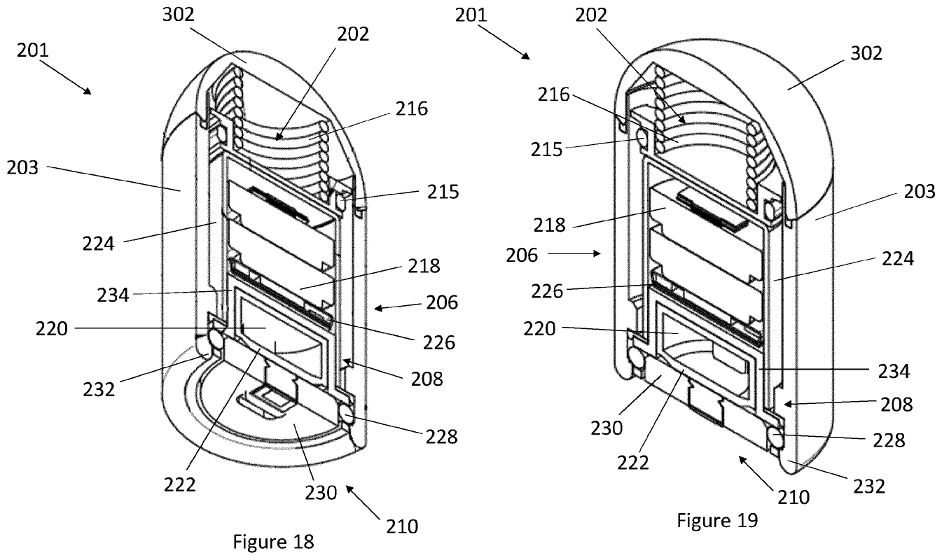

A releasable module 206 is located within the housing 203 and retained there by a retaining mechanism 208 seen in FIG. 17 (and also FIGS. 18, 19, 20 and 27). In use, the releasable module 206 exits the housing 203 through the first opening 210 once released by the retaining mechanism 208, this movement being represented by arrow 212. The intestinal capsule assembly 201 is shown in FIG. 15 in a first, pre-activation configuration in which the releasable module 206 is located within the housing 203. In use, a sample of intestinal fluid is collected through the first opening 210 into the sample chamber 202, along the fluid flow path 300 as represented in FIG. 16.

As is seen in FIGS. 17, 18, and 19, the intestinal capsule assembly 201 according to the second embodiment includes all the same components as those of the first embodiment (labelled with like reference numerals, with the value of said corresponding reference numerals increased by 200), with the exception of the second opening and associated features as discussed below.

The intestinal capsule assembly 201 is shown in greater detail in FIG. 17-20. The sample chamber includes the piston 214 and a spring 216 functioning as described above. An O-ring 215 surrounds the piston 214, forming a partial seal against the housing 203, as described in further detail below.

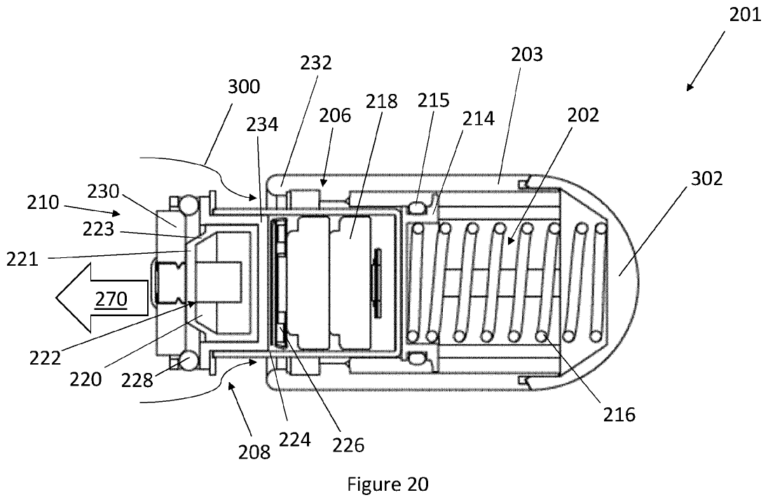

The releasable module 206, includes a battery 218 (made up of two individual cells in the illustrated example) and a fluid container 220 contained within a housing 224, the fluid container 220 including a diaphragm 222, including a flat portion 221 and a deformable webbing portion 223 (seen most clearly in FIG. 20), which function in the same manner as described above with reference to the first embodiment. The releasable module 206 further contains electronic circuitry 226, again as described above.

The releasable module 206 also includes the retaining mechanism 208, including ball locks 228, a release member 230 and a surround 234. In the pre-activation configuration seen in FIG. 17, the ball locks 228 are arranged in frictional engagement between a lip 232 formed around the first opening 210, and the release member 230. The retaining mechanism 208 operates as described above with respect to the first embodiment, such that movement of the diaphragm 222 pushes the release member 230 along the axial direction, away from the rest of the releasable module 206, as represented by the arrow 270 in FIG. 20.

The intestinal capsule assembly 201 does not include a second opening, but instead includes a solid cap 302, which in the illustrated example is rounded. As shown, one end of the spring 216 is biased against the inner side of the cap 302.

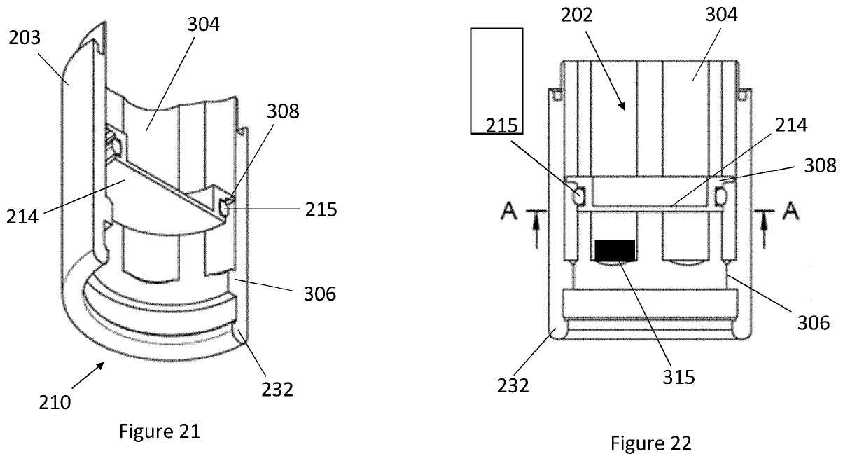

In contrast to the first embodiment, in the second embodiment the fluid flow path 300 along which fluid is drawn into the sample chamber 202 passes through the first opening 210, as seen in FIG. 20. The differences of the second embodiment (and a similar third embodiment), which make this possible, are represented in more detail in FIGS. 21-26.

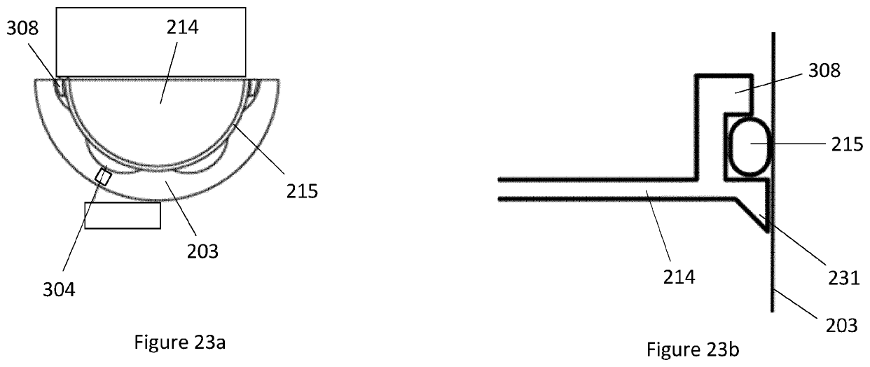

FIGS. 21-23b show various views of the sample chamber 202 of the intestinal capsule assembly 201 according to the second embodiment. FIGS. 21 and 22 show perspective views of the inside of the housing 203, with certain components (e.g. the spring 216) omitted to more clearly show certain features. The inner surface of the housing 203 includes grooves 304 i.e. elongate depressions in the inner surface of the housing 203. These grooves 304 can also be seen in the cross-sectional view of FIG. 23a (along the line A-A as shown in FIG. 22), which shows the housing 203, surrounding the piston 214, and O-ring 215.

As seen most clearly in FIG. 23a, as a result of these grooves 304, the piston 214 (or surrounding O-ring 215) is not in physical contact with the inner surface of the housing 203 around its entire circumference/perimeter. As a result, as the piston 214 moves in the direction 212, as illustrated in FIG. 15, fluid is drawn in along the grooves 304, along the fluid flow path 300, through the space which is left between the inner surface of the housing 213 and the piston 214, i.e. the space defined by the grooves 304.

In order to seal the sample chamber 202 once sample collection is complete, the inner surface of the housing 203 additionally comprises a restricted portion 306. The restricted portion 306 is formed by the inner surface of the housing 203 protruding inwards (i.e. towards the centre of the housing) just below the lower end (closest the first opening 210) of the grooves 304, to form a region having a circumference approximately equal to that of the piston 214 and O-ring 215 together, against which the O-ring 215 seals.

The piston 214 additionally comprises tabs 308, which extend outwards beyond the circumference of the rest of the piston 214. As seen in the cross-section of FIG. 23a, these tabs 308 are located on opposing sides of the piston 214 and extend into two of the (plurality of) grooves 304 to guide the movement of the piston 214 as it slides within the housing 203. The lower end of the grooves 304 may protrude inwards, forming surfaces which act as a stop, stopping movement of the tabs 308, and therefore the piston 214, any further.

As shown in the cross-sectional side view of FIG. 23b, the piston 214 may include a sharp edge 231 on its lower side, i.e. the side of the piston 214 which is closer to the first opening. When the piston 214 reaches the restricted portion 306 this sharp edge 231 will help to shear material that has partially entered the grooves 304, thus helping to clear the path for the piston 214 to reach maximum, or end position e.g. contacting the stop at the end of the grooves 304.

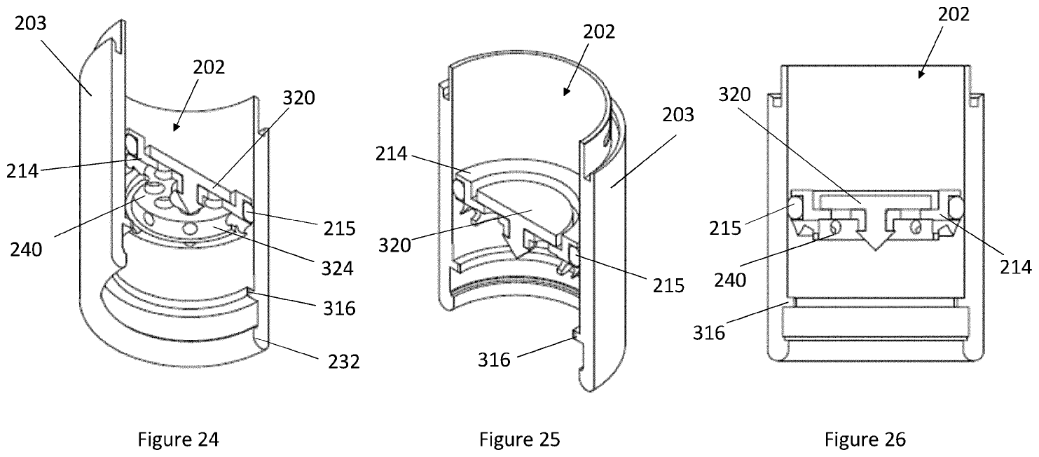

FIGS. 24-26 show various views of a sample chamber of an intestinal capsule assembly according to a third embodiment, having all the same features as the intestinal capsule assembly of the second embodiment, apart from the grooves 304 as illustrated in FIGS. 21-23b.

Instead of these grooves, passage of fluid into the sample chamber 202 is made possible by the piston 214 comprising a one-way valve 320. The one-way valve 320 only allows passage of fluid into the sample chamber 202, i.e. such that as the piston moves in the direction of the schematic arrow seen in FIGS. 24-26 fluid is drawn through the one-way valve 320 in the direction opposite to the arrow, but the valve does not allow passage of fluid back out of the chamber.

The outer surface of the O-ring 215 and piston 214 is sealed against the smooth inner wall of the housing 203, so that the only opening through which fluid might pass is the one-way valve 320. There is again a restricted portion 316, separate to lip 232, which in this case both provides a stop, against which movement of the piston 214 is stopped at the end of sample collection, and a restriction against which the O-ring 215 is sealed. In this embodiment the stop 316 is formed as a circumferential lip around the inner surface of the housing. As shown, it is above (i.e. further inside the housing than) the lip 232 against which the locking members are biased in the pre-activation configuration.

In the illustrated example, the piston 214 further comprises a filter 240. This filter 240 is arranged to cover the one-way valve 320, with the filter 240 being below the one-way valve 320 according to the view of FIGS. 24-26 (i.e. closer to the first opening so that the filter 240 is further upstream with respect to fluid passing through the valve 320). The filter 240 is intended to induce shear in viscous fluid drawn through the filter 240. The filter 240 may comprise openings formed with sharp edges. It may comprise any of the features described above with reference to filters 40 of the first embodiment.

In either the second or third embodiment described above a dissolvable blocking member may be placed within the housing 203, arranged to prevent movement of the piston 214 to a position in which the sample chamber 202 is sealed. For example, the dissolvable blocking member may be arranged in a groove of the inner wall of the housing, as illustrated by the dissolvable blocking member 315 shown in FIG. 22. After some time of exposure to gut fluids entering the sample chamber along the fluid flow path the blocking member will be dissolved by these fluids, and closing of the sample chamber 202 will be possible.

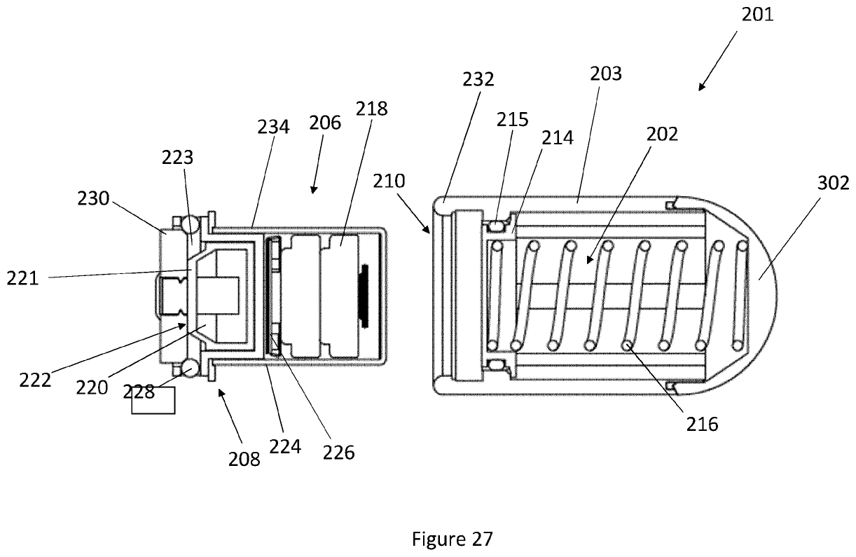

FIG. 27 shows the intestinal capsule assembly 201 in a post-activation configuration in which the releasable module 206 is fully outside of the housing 203.

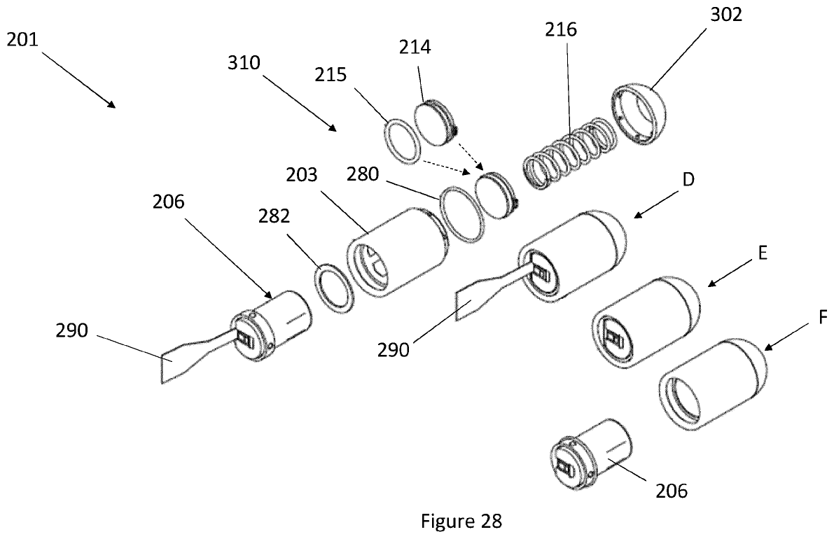

FIG. 28 then shows an exploded view of the components included in the assembled intestinal capsule assembly 201. It can be seen that the intestinal capsule assembly 201 includes a first gasket 280, arranged between the housing 203 and the cap 302. The piston assembly 310 is made up of the piston 214, the O-ring 215, and the housing 203. The intestinal capsule assembly 201 also includes a second gasket 282, arranged between the releasable module 206 and the housing 203. In FIG. 28 the intestinal capsule assembly 201 is shown first in the inactivated condition, labelled as D, in which the pull tab 290 is preventing battery connection. In the pre-activation condition, labelled as E, in which the pull tab 290 has been removed, the intestinal capsule assembly 201 is ready to be swallowed by a subject. FIG. 28 further shows the intestinal capsule assembly 201 in the post-activation condition, labelled as F, in which the releasable module 206 has fully exited the housing 203, as shown in cross-section in FIG. 27.

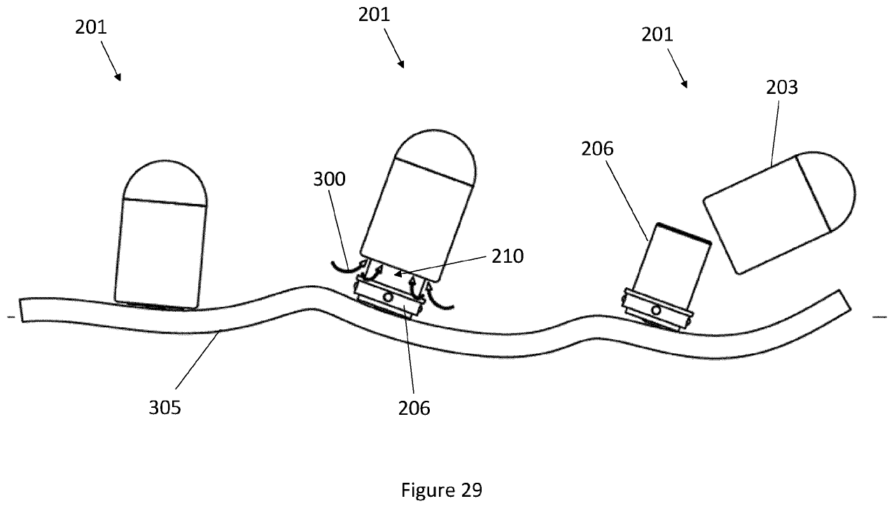

An advantage of the intestinal capsule according to the second and third embodiments of the invention, as described above, is illustrated with reference to FIG. 29, which shows the stages of sample collection using a capsule assembly according to a second or third embodiment of the present invention. The left hand side of FIG. 29 shows the intestinal capsule assembly 201 in the pre-activation condition, in which it happens to be positioned against the gut wall 305. The central part of FIG. 29 shows the intestinal capsule assembly 201 during sample aspiration, where a sample is being drawn in along the fluid flow path 300, as represented by four of arrows. The right hand side of FIG. 29 then shows the intestinal capsule assembly 201 in the post-activation condition, in which the releasable module 206 has fully exited the housing 203.

Generally, as fluid is being drawn into a capsule within the gut, there is a risk that the action of drawing in the fluid creates a suction force between the capsule and the gut wall, causing the capsule to be suctioned onto i.e. against, the gut wall, if the whole opening through which the sample is being drawn comes into contact with the gut wall, creating a seal. The intestinal capsule assembly 201 according to the second and third embodiments is particularly effective at preventing this as seen in FIG. 29, since the ejection of the releasable module 206 pushes the housing 203, including the first opening 210, away from the gut wall during ejection. This prevents the gut wall 305 from coming into close contact with the first opening 210, thus minimizing the risk of having the intestinal capsule assembly suctioning onto the side wall of the gut of a subject during sample aspiration. Furthermore, if the intestinal capsule assembly 201 happens to end up in any other configuration with respect to the gut wall 305, then it will be the closed housing 203, or the closed, i.e. solid cap 302, which is in contact with the gut wall 305, and there will thus be no risk of suctioning against the gut wall 305.

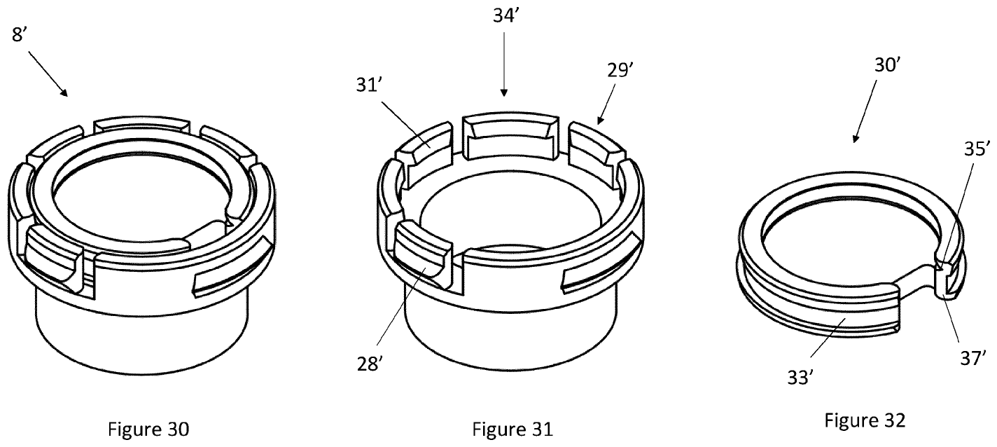

FIGS. 30 to 35 show an alternative retaining mechanism 8′ which may be used in place of the retaining mechanism 8 in any of the embodiments described above. This retaining mechanism 8′ is made up of a surround 34′ (shown in FIG. 31) and a release member 30′ (shown in FIG. 32), which is placed within the surround 34′ (in the position shown in FIG. 30).

The surround 34′ includes locking members in the form of external ridges 28′ (i.e. ridges 28′ which extend radially from the outside surface of the surround 34′) which are arranged on respective tabs 29′, extending axially from the main body of the surround 34′. Alternatively, a single continuous e.g. circumferential, ridge may be provided. The surround 34′ (and in this example specifically the tabs 29′) also comprise internal ridges 31′, which extend radially towards the inside of the surround 34′.

The release member 30′ includes a circumferential groove 33′, which is defined by an upper ledge 35′ and a lower ledge 37′, each extending circumferentially around the release member 30′ and extending radially outwards from it.

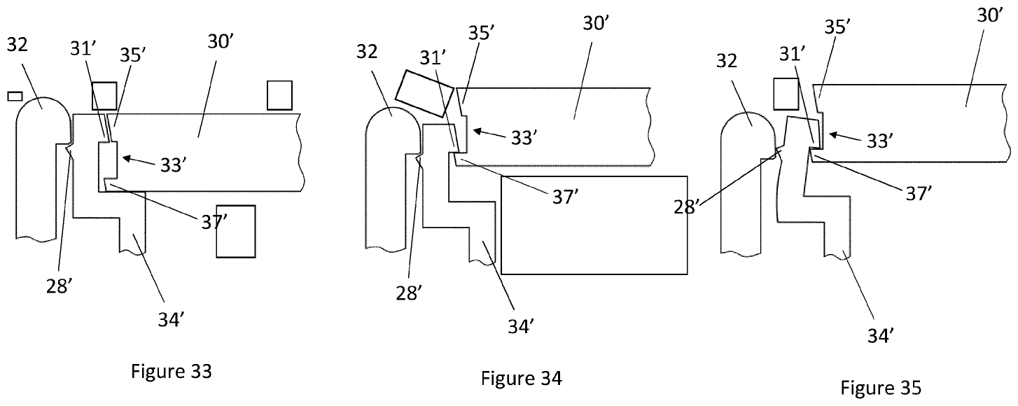

The process of release of the releasable module by the retaining mechanism 8′ will now be described with reference to FIGS. 33 to 35.

In the pre-activation position, as shown in FIG. 33, the external ridges 28′ (i.e. locking members) of the surround 34′ are arranged in frictional engagement between a lip 32 formed around the first opening and the release member 30′. This position can also be referred to as the engaging position. In this position, the upper ledge 35′ of the release member 30′ is pressed into frictional engagement with the internal ridge 31′ of the surround 34′, which prevents movement of the external ridge 28′ out of the engaged position. In other words, in this engaging position the external ridges 28′ (i.e. locking members) are engaged with the housing to retain the releasable module within the housing.

The release member 30′ is arranged to be moved by the diaphragm 22′ (not shown), which is moved by the gas pressure generated in the fluid container 20′. This moves the release member 30′ axially upwards (from the point of reference of FIGS. 33 to 35) into the position shown in FIG. 34. The position shown in FIG. 34 is a non-engaging position. This movement causes the upper ledge 35′ of the release member 30′ to no longer engage with the internal ridges 31′ of the surround 34′. Instead, the internal ridges 31′ are aligned (in the axial direction) with the circumferential groove 33′ of the release member 30′. As a result, the internal ridges 31′ can move radially inwards, and therefore so can the external ridges 28′, thus the external ridges 28′ (i.e. locking members) are no longer engaged so as to retain the releasable module. In this position the releasable module is therefore moveable relative to the housing. Once the release member 30′ has moved further upwards, the lower ledge 37′ of the release member 30′ contacts the internal ridges 31′ of the surround 34′. This prevents the release member 30′ from moving completely outside of the surround 34′, and keeps the components together even once the releasable module is released from the assembly.

The surround 34′ is preferably made of a material which has some flex or bend. As a result, the surround 34′ can bend radially inwards, as is shown in FIG. 35. This allows the releasable module to be fully released even if the opening defined by the lip 32 is slightly smaller than the circumference of the surround 34′, i.e. its “natural” circumference in a neutral and unstressed position. This bending or flexing may be helped by a spring or gas pressure force acting on the releasable module to help it move out of the housing, as described above.

This retaining mechanism 8′ provides an energy-efficient mechanism, capable of controlling the strong compression spring needed for aspirating viscous fluid into the sample chamber.

It will be appreciated by those skilled in the art that the invention has been illustrated by describing one or more specific embodiments thereof, but is not limited to these embodiments; many variations and modifications are possible, within the scope of the accompanying claims.

1. An intestinal capsule assembly, comprising:
a housing, the housing comprising a first opening, and a sample chamber defining a volume for collecting a sample of intestinal fluid;
a releasable module, comprising a battery and a fluid container; and
a retaining mechanism, for retaining the releasable module within the housing such that the releasable module closes the first opening;
wherein the intestinal capsule assembly has a first, pre-activation configuration in which the releasable module is located within the housing and a second, post-activation configuration, in which the releasable module is located outside of the housing; and
wherein the releasable module is arranged to be activated, to cause the battery to supply power to the fluid container so as to cause, in use, electrolysis of a fluid within the fluid container to create a gas pressure, wherein the gas pressure causes release of the retaining mechanism, so as to allow movement of the releasable module out of the housing through the first opening, resulting in expansion of the volume of the sample chamber.

2. The intestinal capsule assembly of claim 1, further comprising a piston, wherein the piston defines a wall of the sample chamber and the expansion of the volume of the sample chamber causes movement of the piston towards the first opening and wherein the housing is arranged to stop the piston from moving out of the housing through the first opening.

3. (canceled)

4. The intestinal capsule of claim 2, wherein the piston is resiliently biased and wherein the piston provides an ejection mechanism for the releasable module which applies a force to the releasable module which results in the releasable module being ejected from the housing through the first opening, moving the intestinal capsule assembly to the post-activation configuration.

5. The intestinal capsule assembly of claim 2, further comprising a flexible attachment, connecting the piston and the releasable module.

6. The intestinal capsule assembly of claim 1, wherein the sample chamber comprises gas generation means, arranged to be triggered, in use, by a sample of intestinal fluid within the sample chamber, to generate gas.

7. (canceled)

8. (canceled)

9. (canceled)

10. The intestinal capsule assembly of claim 1, wherein the releasable module is arranged to be remotely activated, for example by magnetic field triggering, magnetic near-field induction, radio waves, ultrasonic coupling communication, or capacitive coupling communication, or galvanic coupling communication.

11. The intestinal capsule assembly of claim 1, wherein the sample chamber comprises a preservative for preserving certain properties of a sample of intestinal fluid collected in use.

12. The intestinal capsule assembly of claim 1, wherein the assembly comprises a laxative, which is released as a result of release of the retaining mechanism.

13. The intestinal capsule assembly of claim 1, wherein an outer surface of the housing is at least partly coated with an ultrasound contrast agent.

14. The intestinal capsule assembly of claim 1, wherein the housing further comprises a second opening and the sample chamber is arranged for collecting a sample of intestinal fluid through the second opening, wherein the housing comprises at least one filter arranged to cover the second opening, and wherein the at least one filter is configured to induce shear in the intestinal fluid as the fluid passes through the at least one filter, so as to reduce the viscosity of the intestinal fluid before the intestinal fluid reaches the second opening.

15. (canceled)

16. The intestinal capsule assembly of claim 14, wherein the second opening and/or the at least one filter comprises an enzymatic coating which reduces viscosity of the intestinal fluid.

17. (canceled)

18. An intestinal capsule assembly, comprising:
a housing, comprising a first opening, and a sample chamber defining a volume for collecting a sample of intestinal fluid along a fluid flow path, wherein the fluid flow path passes through the first opening;
a releasable module; and
a retaining mechanism, for retaining the releasable module within the housing such that the releasable module closes the first opening;
wherein the intestinal capsule assembly has a first, pre-activation configuration in which the releasable module is located within the housing and a second, post-activation configuration, in which the releasable module is located outside of the housing; and
wherein the releasable module is arranged to be activated, so as to cause release of the retaining mechanism, so as to allow movement of the releasable module out of the housing through the first opening, allowing a sample of intestinal fluid to be collected along the fluid flow path.

19. (canceled)

20. The intestinal capsule assembly of claim 18, wherein the intestinal capsule assembly further comprises a piston, wherein the piston defines a wall of the sample chamber and the expansion of the volume of the sample chamber causes movement of the piston towards the first opening; and wherein the piston comprises at least one tab, arranged to be positioned within a groove or indent of the inner wall of the housing when the piston abuts against the inner wall of the housing.

21. (canceled)

22. The intestinal capsule assembly of claim 18, wherein the intestinal capsule assembly comprises a dissolvable blocking member, arranged to prevent movement of the piston to a position in which the sample chamber is sealed.

23. The intestinal capsule assembly of claim 18, wherein an external edge of the piston comprises a sharp edge.

24. An intestinal capsule assembly comprising a sample chamber, for collecting a sample of intestinal fluid, the sample chamber comprising an opening through which the sample is collected,
wherein the sample chamber comprises at least one filter arranged to cover the opening, wherein the at least one filter is configured to induce shear in the intestinal fluid as the fluid passes through the filter, so as to reduce the viscosity of the intestinal fluid before the intestinal fluid reaches the opening.

25. The intestinal capsule assembly of claim 24, wherein the at least one filter comprises slots, and at least one of:
at least one sharp edge; and/or
small openings, wherein the openings are misaligned with the slots and/or wherein the openings are smaller than the slots;
arranged so that fluid passing through the slots then passes over the sharp edge and/or through the openings, to shear the fluid from the slots.

26. The intestinal capsule assembly of claim 24, wherein the at least one filter comprises an outer filter, and wherein the outer filter is curved or bent, such that intestinal fluid can be collected from different directions.

27. An intestinal capsule product, comprising an intestinal capsule assembly as claimed in claim 1 and a sterile packaging;
wherein the intestinal capsule assembly further comprises a pull tab, arranged to obstruct an electrical flow path from the battery, wherein the pull tab comprises at least one electronic connection; and
wherein the intestinal capsule assembly is at least partially retained within the sterile packaging, such that the at least one electronic connection extends beyond the sterile packaging.

28. A product comprising an electronic medical device, and a sterile packaging, at least partly enclosing an intestinal capsule assembly as claimed in claim 1, the intestinal capsule assembly comprising:
a battery; and
at least one electronic connection;
wherein the packaging is arranged such that the at least one electronic connection extends outside of the packaging.

29. (canceled)

30. (canceled)

31. (canceled)
